(19) United States
(12) Patent Application Publication
(10) Pub. No.: US 2023/0190980 A1
(43) Pub. Date: Jun. 22, 2023

(54) AIRBORNE INFECTIOUS AGENT STERILIZER AND RELATED METHOD

(71) Applicant: Claudio FILIPPONE, College Park, MD (US)

(72) Inventor: Claudio FILIPPONE, College Park, MD (US)

(21) Appl. No.: 18/165,279

(22) Filed: Feb. 6, 2023

Related U.S. Application Data
(63) Continuation of application No. PCT/US2021/053441, filed on Oct. 4, 2021.
(60) Provisional application No. 63/108,868, filed on Nov. 2, 2020.
(60) Provisional application No. 63/090,278, filed on Oct. 11, 2020.
(60) Provisional application No. 63/060,751, filed on Aug. 4, 2020.

(51) Int. Cl. A61L 9/18; B60H 3/00

(52) U.S. Cl. CPC A61L 9/18; B60H 3/0071; A61L 2209/134; A61L 2209/16; A61L 2209/11

(57) ABSTRACT

A device includes a housing including an inlet and an outlet, the inlet being configured to intake an infectious agents contaminated air, and the outlet being configured to output a sterilized air. The device also includes a superheating heat exchanger configured to increase a temperature of the infectious agents contaminated air by superheating the contaminated air, the infectious agents contaminated air becoming the sterilized air after being superheated. The device further includes a cooling heat exchanger configured to cool down the sterilized air and direct the sterilized air to the outlet of the housing.

### CROSS-REFERENCE TO RELATED APPLICATIONS

This application is a continuation of International Application No. PCT/US2021/053441, filed on Oct. 4, 2021, which claims priority to U.S. Provisional Application No. 63/060,751, filed on Aug. 4, 2020, U.S. Provisional Application No. 63/090,278, filed on Oct. 11, 2020, U.S. Provisional Application No. 63/108,868, filed on Nov. 2, 2020. The contents of all of the above-mentioned applications are incorporated herein by reference in their entirety.

### TECHNICAL FIELD

The present disclosure relates generally to air sterilizing device and method and, more specifically, to an airborne infectious agent sterilizer and related method.

### BACKGROUND

Severe Acute Respiratory Syndrome (SARS), is an emerging disease associated with severe pneumonia. Novel coronavirus causes SARS with a dramatic impact on health care services and economies of affected countries with a high mortality rate. A notable feature of the coronavirus is its predilection for transmission in the health care setting as well in closed environments and via social contacts. Some virus spreading mechanisms include airborne droplets suspended and carried by environmental air, close direct contact and indirect contact (e.g., contact with contaminated surfaces). Airborne droplet transmission is one of the means of transmission.

### SUMMARY OF THE DISCLOSURE

Consistent with an aspect of the present disclosure, a device is provided. The device includes a housing including an inlet and an outlet, the inlet being configured to intake an infectious agents contaminated air, and the outlet being configured to output a sterilized air. The device also includes a superheating heat exchanger configured to increase a temperature of the infectious agents contaminated air by superheating the contaminated air, the infectious agents contaminated air becoming the sterilized air after being superheated. The device further includes a cooling heat exchanger configured to cool down the sterilized air and direct the sterilized air to the outlet of the housing.

Consistent with another aspect of the present disclosure, a device for sterilizing air in a vehicle is provided. The device includes a recuperator heat exchanger configured to increase a temperature of an infectious agents contaminated air including airborne infected agents to sterilize the infectious agents contaminated air as a sterilized air. The device also includes a heat source heat exchanger configured to exchange thermal energy with a heat source and provide the thermal energy to the recuperator heat exchanger. The device further includes a controller configured to control an air flow of the contaminated air, and the temperature of the contaminated air increased to by the recuperator heat exchanger.

Other aspects of the present disclosure can be understood by those skilled in the art in light of the description, the claims, and the drawings of the present disclosure.

### BRIEF DESCRIPTION OF THE DRAWINGS

The following drawings are provided for illustrative purposes according to various disclosed embodiments and are not intended to limit the scope of the present disclosure. In the drawings:

FIG. 1 is a schematic illustration of an airborne infectious agents sterilizer (may also be referred to as a virus neutralizer apparatus, device, or system, or airborne infectious agents neutralizer apparatus, device, or system; for simplicity, may be referred to as air sterilizer), according to an embodiment of the present disclosure, illustrating the main components integrated within a housing including heating mechanisms and equipped with an inlet and an outlet for contaminated air to go through the sterilizer for de-contamination treatment.

FIG. 2A is a block diagram illustrating open-loop thermodynamic cycles experienced by the contaminated air as it flows through the air sterilizer, according to an embodiment of the present disclosure.

FIG. 2B is a representative temperature plot indicating different thermodynamic states experienced by the contaminated air as it flows through various stages forming the air sterilizer, according to an embodiment of the present disclosure.

FIG. 3 is a schematic illustration of an air sterilizer shown in FIG. 1, according to an embodiment of the present disclosure, illustrating the main components integrated within a housing, the main components further including Ultra-Violet (UV) sanitizing lamps and internal mirrors, and the housing being equipped with an inlet and an outlet for contaminated air to go through for air sterilization processes.

FIG. 4 a schematic illustration of an air sterilizer shown in FIG. 1 and FIG. 3, according to an embodiment of the present disclosure, further illustrating the main control system components integrated within a housing, the main components including features for Bluetooth connection to external devices (e.g., smart phones, computers), electric power supply (e.g., battery pack and external), a communication system equipped with microphone and speakers to communicate with other individuals wearing specialized air sterilizer masks of the present disclosure and sensors/data utilized by the controller to ensure contaminated air is treated according to sterilization requirements.

FIG. 5 is a schematic illustration of an air sterilizer shown in FIG. 1, FIG. 3 and FIG. 4, according to an embodiment of the present disclosure, further illustrating additional features configured to treat a user's exhaled air (potentially infected) prior to venting it to atmosphere so as to sterilize the air inhaled by the user.

FIG. 6 is a schematic illustration of the air sterilizer shown in FIG. 5 further equipped with an auxiliary compressor/circulator and natural convection cooling fins thermally coupled to environmental air outside of the air sterilizer and the air being treated internally to the air sterilizer, according to an embodiment of the present disclosure.

FIG. 7 is a schematic illustration of the air sterilizer shown in FIG. 6, further equipped with an auxiliary cooling loop compressor/circulator and forced convection cooling through a stream of contaminated environmental air thermally coupled with the air treated after user's exhalation so as to lower the temperature of the vented sterilized air, according to an embodiment of the present disclosure.

FIG. 8 is a scalable schematic illustration of the air sterilizer shown in FIGS. 1-7 to treat large volumes of, for example, virus-contaminated air, according to an embodiment of the present disclosure, further equipped with an auxiliary heating loop to increase the temperature of the contaminated air during processing within the air sterilizer, and a cooling closed- or open-loop with dedicated compressor/circulator and forced convection cooling fluid thermally coupled to treated air to lower its temperature prior to venting sterilized air within a controlled volume (e.g., hospitals, factories, offices, indoor environment and vehicles cabins).

FIG. 9 is a scalable schematic illustration of the air sterilizer shown in FIGS. 1-8 according to an embodiment of the present disclosure, configured to treat large volumes of contaminated air, further equipped with an auxiliary air-conditioning system to cool treated air and lower its temperature prior to venting sterilized air within a controlled volume (e.g., hospitals, factories, offices, indoor environments).

FIGS. 10A-10C illustrate perspective and transparent views of a configuration of the air sterilizer shown in FIGS. 1-7, according to an embodiment of the present disclosure, showing contaminated air at the inlet through filter, compressor, heating and cooling internals and the sterilized air at the outlet.

FIGS. 11A-11C illustrate perspective, semi-transparent and cross-sectional views of the heating cartridge according to an embodiment of the present disclosure, including electrical heaters embedded with a heat exchanger configured to superheat contaminated air flowing through clearances and fins thermally coupled with the electrical heaters.

FIGS. 12A-12D illustrate a perspective and semi-cross-sectional views of the air sterilizer according to an embodiment of the present disclosure, including UV lamps, configured with internal clearances and mirrors for the UV light to resonate within the air sterilizer housing and through which contaminated air flows prior to inletting the super-heating portions of the air sterilizer.

FIGS. 13A and 13B illustrate a perspective view of a superheating cartridge according to an embodiment of the present disclosure, configured to enable flow and exposure of contaminated air to extended high-temperature surfaces, further equipped with cooling fins thermally coupled with environmental air to cool down the superheated air at the outlet of the superheating cartridge.

FIGS. 14A and 14B illustrate a configuration of the air sterilizer according to an embodiment of the present disclosure, with the components fully integrated with a wearable mask enabling sterilization of virus-contaminated air prior to inhalation and prior to venting exhaled air back to the environment for operation of the mask within potentially infected indoor/closed environments.

FIGS. 15A-15C illustrate another configuration of the air sterilizer according to an embodiment of the present disclosure, with the components integrated within a “back-pack unit” wearable enclosure to accommodate for larger battery supply, showing the sterilized virus-free air hydraulically provided to a wearable mask for inhalation, and exhaled air hydraulically coupled to the back-pack unit for processing of the exhaled air prior to venting it to the environment for operator within closed environments possibly housing multiple individuals.

FIGS. 16 illustrates a wearable “belt-unit” configuration of the air sterilizer described in FIGS. 1-7 with flexible tubing coupling the belt-unit equipment to the wearable mask according to an embodiment of the present disclosure.

FIG. 17 illustrates a wearable “arm-unit” configuration of the air sterilizer described in FIGS. 1-7 according to an embodiment of the present disclosure, with flexible tubing coupling the belt-unit equipment to the wearable mask.

FIG. 18 illustrates a scaled-up configuration of the air sterilizer described in FIGS. 8 and 9 according to an embodiment of the present disclosure for applications processing large volumes of contaminated air with integrated cooling system to condition the sterilized air prior to venting it within the controlled volume.

FIG. 19 illustrates a scaled-up configuration of the air sterilizer described in FIGS. 8, 9, and 18 according to an embodiment of the present disclosure for applications processing large volumes of contaminated air by coupling the air sterilizer with standard air-conditioning equipment to condition the superheated air prior to venting it within a controlled volume.

FIG. 20 is a schematic illustration of a scalable air sterilizer, according to an embodiment of the present disclosure, illustrating the main components integrated within a housing including heating mechanisms and equipped with a contaminated air inlet and at least one sterilized air outlet for contaminated air to go through for sterilization.

FIG. 21 is a functional schematic diagram illustrating the environmental control and air-conditioning system of a passenger railcar, in which environmental air flows through filters and air conditioning heat exchangers prior to being distributed inside the railcar cabin.

FIG. 22 is a functional schematic diagram of an Environmental Control Systems(ECS) and air-conditioning system of the railcar shown in FIG. 21 with integrated air sterilizer retrofitted with the ECS and air-conditioning components, according to an embodiment of the present disclosure.

FIG. 23 is a schematic illustration of the scalable air sterilizer shown in FIGS. 20-22, according to an embodiment of the present disclosure, configured to treat contaminated air circulating within the closed environment represented by a passenger railcar, or any other high-occupancy vehicle .

FIG. 24 illustrates a perspective view of a configuration of the air sterilizer shown in FIGS. 20-22, according to an embodiment of the present disclosure, showing contaminated-air intake nozzles.

FIG. 25 illustrates a perspective views of sterilized air curtains output from sterilized air outlet nozzles of the air sterilizer and the range of contaminated air capture by contaminated air intake nozzles of the air sterilizer, according to an embodiment of the present disclosure.

FIG. 26 illustrates a perspective view of the sterilized air curtains provided by the air sterilizer by locating the sterilized air outlet nozzles and the contaminated air intake nozzles in the context of passengers seats (e.g., railcars, airplane cabin, bus etc.), according to an embodiment of the present disclosure.

FIG. 27 illustrates a perspective frontal view of the sterilized air outlet nozzles, and the contaminated air intake nozzles, according to an embodiment of the present disclosure.

FIG. 28 illustrates a configuration of the auxiliary sterilized air nozzle distributors and the contaminated air intake nozzles of the air sterilizer, according to an embodiment of the present disclosure, further showing the high-temperature waste heat recovery fluid inlet and outlet in the context of a railcar ECS and heating, ventilation, and air conditioning (HVAC) system.

FIG. 29 illustrates a configuration of the air sterilizer shown in FIG. 28, according to an embodiment of the present disclosure, in which the waste heat thermal source utilized to superheat the contaminated air is represented by a heat exchanger thermally coupled to the vehicle (e.g., locomotive) exhaust gases, and the working fluid utilized to transfer thermal energy from the exhaust gases coupled to the air sterilizer auxiliary thermal energy inlet and outlet ports.

FIG. 30 illustrates an embodiment of the air sterilizer retrofitted with the ECS and air-conditioning system equipping the fuselage/cabin of aircrafts, according to an embodiment of the present disclosure.

FIG. 31 illustrates the distribution of sterilized air outlet nozzles and contaminated air intake nozzles surrounding passenger seats in the context of the seats arrangement in the cabin of an aircraft, according to an embodiment of the present disclosure.

FIG. 32 illustrates sterilized air curtains surrounding passengers within the fuselage of an aircraft equipped with the air sterilizer shown in FIGS. 30-31, according to an embodiment of the present disclosure.

FIG. 33 shows an embodiment of the air sterilizer retrofitted with the ECS and air-conditioning system equipping the cabin of a bus, according to an embodiment of the present disclosure.

FIG. 34 is a top-cross sectional view of a bus equipped with air conditioning, air handling and HVAC systems to control the temperature of the “comfort air” for passengers and operators' consumptions.

FIG. 35 is a top-cross sectional view of the bus shown in FIG. 34 with multiple air sterilizers retrofitted with the air conditioning, air handling and HVAC systems to sterilize the air circulating within the vehicle prior to being vented by the vehicle's comfort air vents, according to an embodiment of the present disclosure.

FIG. 36 is a functional diagram illustration of the main components forming an operational air sterilizer according to an embodiment of the present disclosure.

FIG. 37 is a 3-D representation of the scalable air sterilizer illustrating the main components forming the recuperator heat exchanger, a heat source, and a compressor to circulate potentially infected air to sterilize it as it flows through the recuperator heat exchanger and the heat source, according to an embodiment of the present disclosure.

FIG. 38 is a photographic representation of one configuration of the hardware of the air sterilizer, where the recuperator heat exchanger is formed by a finned-tube in as an embodiment of the present disclosure.

FIG. 39 is a photographic representation of the fully operational air sterilizer shown in FIG. 20, according to an embodiment of the present disclosure.

FIG. 40 is a photographic view of a different configuration of the operational air sterilizer shown in FIG. 20, according to an embodiment of the present disclosure.

FIG. 41 is a see-through 3D representation of a bus retrofitted with air sterilizer configured to utilize waste thermal energy from the operations of the bus as thermal source to superheat potentially infected air, according to an embodiment of the present disclosure.

FIG. 42 shows two photographic views of the comfort air outlet for passengers and for the driver of a transit bus as sterilized air from the air sterilizer can be distributed to the vehicle for passengers and operators, according to an embodiment of the present disclosure.

FIG. 43 shows two photographic views of the passenger and driver return air vents, where the return air is circulated within the recuperator heat exchanger of the air sterilizer for sterilization prior to be processed by the HVAC systems on board of the transit bus, according to an embodiment of the present disclosure.

FIG. 44 schematically illustrates a simplified passenger cabin HVAC ducting system normally equipping cruise ships.

FIG. 45 schematically illustrates spreading of contaminated air in the passenger cabin HVAC ducting system shown in FIG. 44.

FIG. 46 schematically illustrates the disclosed contaminated air sterilizer retrofitted directly with the centralized air return duct system of a cabin, according to an embodiment of the present disclosure.

FIG. 47 schematically illustrates scaled air sterilizer retrofitted with the fan and coil unit in each passenger cabin, according to an embodiment of the present disclosure.

### DETAILED DESCRIPTION

Embodiments consistent with the present disclosure will be described with reference to the accompanying drawings, which are merely examples for illustrative purposes and are not intended to limit the scope of the present disclosure. Wherever possible, the same reference numbers are used throughout the drawings to refer to the same or similar parts, and a detailed description thereof may be omitted.

The phrase “at least one of A or B” may encompass all combinations of A and B, such as A only, B only, or A and B. Likewise, the phrase “at least one of A, B, or C” may encompass all combinations of A, B, and C, such as A only, B only, C only, A and B, A and C, B and C, or A and B and C. The phrase “A and/or B” may be interpreted in a manner similar to that of the phrase “at least one of A or B.” For example, the phrase “A and/or B” may encompass all combinations of A and B, such as A only, B only, or A and B. Likewise, the phrase “A, B, and/or C” has a meaning similar to that of the phrase “at least one of A, B, or C.” For example, the phrase “A, B, and/or C” may encompass all combinations of A, B, and C, such as A only, B only, C only, A and B, A and C, B and C, or A and B and C.

The term “processor” used herein may encompass any suitable processor, such as a central processing unit (“CPU”), a graphics processing unit (“GPU”), an application-specific integrated circuit (“ASIC”), a programmable logic device (“PLD”), or any combination thereof. Other processors not listed above may also be used. A processor may be implemented as software, hardware, firmware, or any combination thereof

The term “controller” may encompass any suitable electrical circuit, software, or processor configured to generate a control signal for controlling a device, a circuit, an optical element, etc. A “controller” may be implemented as software, hardware, firmware, or any combination thereof. For example, a controller may include a processor, or may be included as a part of a processor.

### Overview

The present disclosure relates to devices and methods for neutralizing airborne infectious (or infective) agents, such as viruses and bacteria in the air, such as coronavirus (e.g., COVID-19). The infective agent typically consists of a nucleic acid molecule in a protein coating, and is able to multiply once in contact with the living cells of a host, such as an animal or a human being. Thus, in some embodiments, the present disclosure relates to a “virus killing” or “virus neutralizing” apparatuses, devices, systems, units, and methods by means of first superheating and then cooling contaminated environmental air prior to being inhaled by a person, (and animals in an indoor environment as for example pets, or animals in a zoo indoor exhibit), and superheating the exhaled air prior to venting it back to the environment. The virus neutralizing apparatus (device, system, or unit) may also be referred to as virus neutralizer, virus neutralizer apparatus (device, system, or unit), infectious agents and virus neutralization apparatus (device, system, or unit), or real-time air sterilizer apparatus (device, system, or unit). The disclosed apparatus (device, system, or unit) may also be referred to as an airborne infectious agents-contaminated air real-time neutralizer or sterilizer apparatus (device, system, or unit), or simply air sterilizer. For simplicity of descriptions, the disclosed apparatus is referred to as the air sterilizer. The air sterilizer effectively neutralizes the virus, bacteria, spores and living microorganisms contained in droplets/aerosols carried by air by exposing it to thermal energy (e.g., by increasing the temperature of the air) as air flows through the air sterilizer. In some embodiments, the present disclosure relates to various virus-, bacteria- and germ-killing apparatuses (or devices) that utilize thermal energy from, for example, electric radiative, conductive, or convective heaters, infrared heaters, laser or electro-magnetic (e.g., microwave) heaters, and/or an ultraviolet light to superheat and neutralize infectious agents including viruses to effectively de-contaminate the air in real-time prior to inhalation. In some embodiments, the devices are provided with scalable features, low-weight, portable, wearable and autonomous (e.g., battery powered), suitable for applications utilizing specialized face masks. In some embodiments, the devices are scaled up to treat viruses, harmful bacteria and micro-organisms contaminating the air in a closed environment (indoor) to supply sterilized air to more than an individual. In some embodiments, the air sterilizers are further scaled up to treat viruses, harmful bacteria, spores and micro-organisms airborne and contaminating the air for applications in closed indoor environments with use of air-conditioning, as virus transmission can largely occur in air-conditioned environments such as those represented by hospitals, hotels, offices, factories. The air sterilizers may be retrofitted with HVAC systems for stationary applications, such as for a building (e.g., hospitals, hotels, offices, factories). The air sterilizers may be retrofitted with HVAC systems for mobile applications, such as for a vehicle of any kind (e.g., locomotive, trucks, cars, ships, airplanes, etc.).

Novel coronavirus can be neutralized by a high temperature, an ultraviolet light, alkaline, or acidic conditions. Human coronaviruses have been shown to survive in PBS (phosphate buffered saline) salt solutions used for cell culture applications and a generic culture medium for multiple days. However, coronavirus can survive only a few hours after drying at a temperature of about 60° C. Further increasing the temperature further reduces the virus survival time duration and probability. When exposed to a temperature of over 100° C. (e.g., sterilization with saturated steam), the virus survival time duration is reduced to seconds and depending on conditions to even a few milliseconds with higher temperatures. A recent study using surrogate coronaviruses (transmissible gastroenteritis virus (TGEV) and mouse hepatitis virus (MHC)) has investigated the effect of the air temperature and the relative humidity on coronavirus survival on surfaces. The survival effects of the environmental temperature and humidity on SARS coronavirus remain unclear. However, by subjecting potentially contaminated air to super-heating with a temperature of over 200° C., coronaviruses, along with other infectious agents including potentially harmful micro-organisms, spores and bacteria are neutralized.

### Applications

In some embodiments, the present disclosure provides a portable virus neutralizing device, which is referred to as a portable air sterilizer or simply the air sterilizer. The portable air sterilizer may include a light-weight superheater and a cooler configured to enable an individual carrying the device to breathe virus-free air. The portable air sterilizer may be powered by a suitable power source, such as a DC (direct current) power supply, an AC (alternating current) power supply, or a battery. That is, the portable air sterilizer may include a power cord connectable to a DC power supply and/or an AC power supply. In some embodiments, the portable air sterilizer may be used as a medical device to treat proportionally larger volumes of potentially virus-contaminated air to enable hospitals and other indoor facilities (hotels, factories) to mitigate and neutralize the spreading of infectious agents and viruses via droplets aerosols transported via air-conditioning systems.

In some embodiments, the portable air sterilizer may be a wearable virus-neutralizer medical apparatus (or device) formed by circulating virus-contaminated air through an air energy increasing mechanism configured to increase the energy content of controlled amounts of air per unit time to elevate its temperature (e.g., by means of electrical heaters, UV or infrared light, lasers, microwave) to kill infectious agents, including any strain of coronaviruses as it mutates over time.

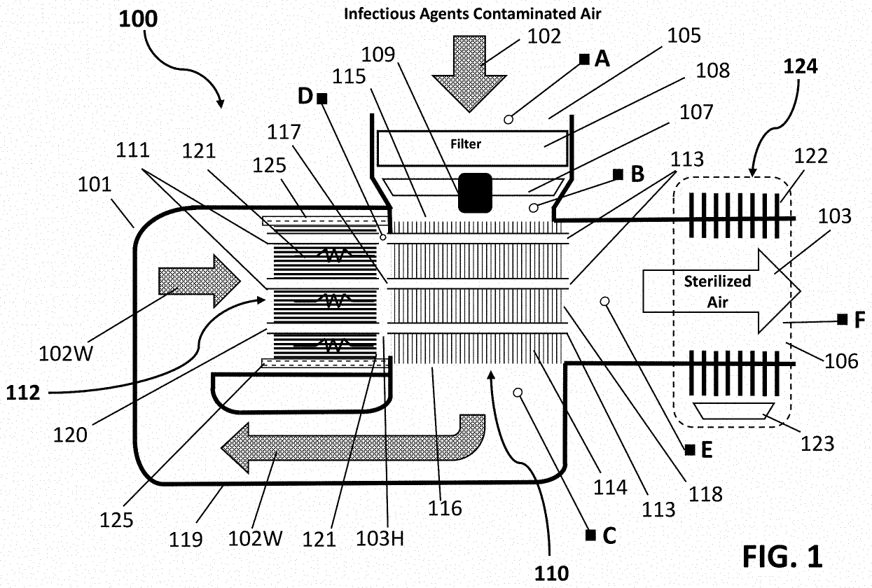

FIG. 1 illustrates an air sterilizer 100 configured to sterilize air in real time via exposing the air to high-temperature surfaces according to an embodiment. FIG. 1 shows the main components integrated within a housing 101 of the air sterilizer 100. The main components include heating mechanisms. The housing 101 is equipped with an inlet 105 and an outlet 106. Infectious agents contaminated air 102 flows from the inlet 105 into the housing 101, and out of the housing 101 through the outlet 106. The contaminated air 102 may be superheated and sterilized as it flows inside housing 101. In some embodiments, the contaminated air 102 may also be treated by additional exposure to UV radiation. The contaminated air 102 may be driven into the inlet 105 by a compressor 107 configured to generate a lower pressure at the inlet side of a filter 108. Thus, the contaminated air 102 may be suctioned into the inlet 105. Particulate and other non-gaseous contaminants contained in the air are filtered by the filter 108, which can be formed by materials generally utilized to form, for example, surgical sterilized masks and other materials generally utilized for air filtering. The compressor 107 may be a single axial turbine compressor, a multistage axial turbine compressor, or a centrifugal radial turbine compressor.

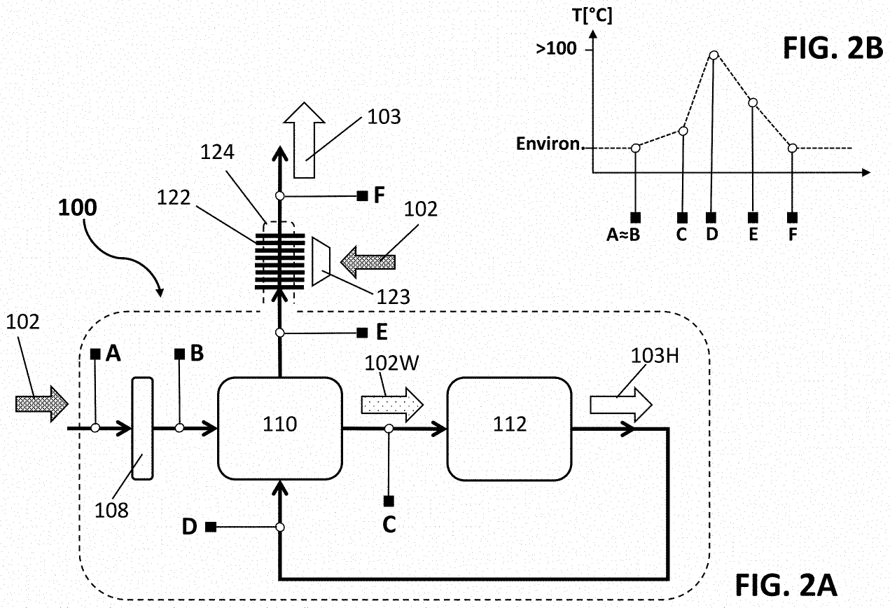

The compressor 107 may be driven by a brushless motor 109. In some embodiments, the compressor 107 may be a positive-displacement mechanical compressor driven by an electric motor (e.g. 109). The compressor 107 may increase the pressure of the contaminated air 102 entering the air sterilizer 100 to overcome a backpressure caused by heat exchangers, fins, channels and clearances disposed within the air sterilizer 100. The heat exchangers, fins, channels and clearances represented are configured to increase time and surface exposure and contact of the contaminated air 102 with the internal hot surfaces as air flows within these components. The contaminated air 102 is subjected to a series of thermodynamic changes prior to being inhaled. The contaminated air 102 is at a thermodynamic state defined by an environmental temperature and pressure denoted as thermodynamic “state A” (shown in FIG. 2B) prior to being compressed by the compressor 107. The contaminated air 102 is at thermodynamic “state B” after being compressed by the compressor 107, and flows to an inlet 115 of a recuperator and cooler heat exchanger 110 (hereafter referred to as “a first heat exchanger 110”). As the contaminated air 102 flows through the first heat exchanger 110, the contaminated air 102 is thermally coupled to the air being processed by a superheating cartridge 112 (hereafter referred to as “a second heat exchanger 112”). The contaminated air 102 is not mixed with sterilized (virus-free) air 103H and 103. The first heat exchanger 110 may be a printed circuit heat exchanger (PCHE), configured with primary channels 114 and secondary channels 113. The contaminated air 102 flows into the inlet 115 of the first heat exchanger 110, where the contaminated air 102 exchanges thermal energy with the superheated sterilized hot air 103H flowing through a secondary side of the first heat exchanger 110. As the contaminated air 102 flows through an outlet 116 at the primary side of the first heat exchanger 110, the thermodynamic state of the contaminated air 102 changes to “state C.” In the “state C,” a temperature of the contaminated air 102 is increased due to heat transfer with the superheated air 103H flowing through an inlet 117 at the secondary side of the first heat exchanger 110. The superheated air 103H may flow through the secondary channels 113 to an outlet 118. In some embodiments, the air sterilizer 100 may operate under a self-sterilization mode. When the air sterilizer is set to self-sterilization mode, the compressor 107 may be operated in a way that the superheated-sterilized air is recirculated inside the air sterilizer 100 so as to increase the temperature of the internal surfaces of the air sterilizer 100 to sterilize any infectious agent that may be depositing or accumulating within the clearances and micro-cracks of the materials from the inlet of the air sterilizer 100 to the outlet of the air sterilizer 100. The self-sterilization mode of operation of the air-sterilizer 100 increases the temperature of all components forming the air-sterilizer 100 above 100° C. for a programmed amount of time to ensure no infectious agent coating the internal surfaces of air sterilizer 100 can proliferate and or cause hazards when the air-sterilizer is disposed or deactivated after periods of operation. When the heaters 121 are on or the combustion gases of a waste heat source is utilized, the fan 107 can be stopped or operated in a way that air inside the device goes back and forth (e.g., by reversing the fan rotation and reversing it again continuously) as a result air inside the device is heated by the heaters, but instead of going out of outlet 106 as the sterilized air 103, the heated air goes backwards toward the inlet. This is done by reversing the fan rotary direction. As air is not really being replaced by fresh contaminated air 102, the internal components of the device superheat, this sterilizing the internal components. This self-sterilization mode can be performed after a certain time of operation (e.g., a day) before shutting it down. This enables the air sterilizer to effectively “cook” all of its internal components (except for the control electronics) so as to sterilize its internals. This prevents infectious agents that made it into the inlet and sticked to the inlet surfaces, and did not make it to the outlet when the air sterilizer is first started and the heaters are still cold for some time. The controller can be programmed to not spin the fan until the internal components are hot, thereby preventing virus and other infectious agents to make it to the user when the air sterilizer is first started and not fully in the sterilization mode.

In some embodiments, the first heat exchanger 110 may be a tube-and-shell type heat exchanger, in which the contaminated air 102 flows through a “shell side” of the corresponding to the primary channels 114, while the superheated air 103H flows through tubes corresponding to the secondary channels 113. In some embodiments, the first heat exchanger 110 may be a compact-finned heat exchanger. The first heat exchanger 110 may lower the temperature of the superheated air 103H from a thermodynamic “state D” to a thermodynamic “state E”, shown in FIG. 2B, while increasing the temperature of contaminated air 102 from a thermodynamic state B to the thermodynamic state C in preparation for superheating through the second heat exchanger 112. In other words, as the contaminated air 102 flows through the first heat exchanger 110, its thermodynamic state changes from state B, to state C as the energy content of the colder contaminated air 102 is increased from the inlet 115 to a contaminated air 102W (Warm) at the outlet 116 as a result of heat transfer from the hotter superheated air 103H (Hot) exiting the second heat exchanger 112. The warmer contaminated air 102W at the thermodynamic state C after exiting the first heat exchanger 110 flows through internal channels 119. The internal channels 119 are formed by a portion of the housing 101. The air 102W may continue to flow through an inlet 120 of the second heat exchanger 112. The air 102W may flow through heating channels 111 (tubes or PCHE channels) and the outlet 118 as the sterilized Hot air 103H at the thermodynamic state D. The second heat exchanger 112 may be a finned, tube or PCHE heat exchanger thermally coupled to electric heaters 121. The second heat exchanger 112 may elevate the temperature of the contaminated air 102W to a predetermined temperature to sterilize infectious agents, including airborne viruses, spores, bacteria and other harmful airborne contaminants, as air flows through the air sterilizer 100. The duty cycle of compressor 107 can be synchronized with breathing cycles to minimize power consumption and maximize breathing comfort. The heaters 121 may be formed by independent clustered resistive elements (e.g., glow plugs), printed circuit heaters, or infrared heaters. The heaters 121 transfer thermal energy to the contaminated air 102W, through convection and radiation, as the contaminated air 102W flows through air-superheating clearances and heat transfer channels formed by materials thermally coupled to the heater 121. As the warm contaminated air 102W flows through the second heat exchanger 112, thermal energy (heat-temperature) transfers from the heaters 121 to the warm, potentially contaminated air 102W to increase its temperature and transform air 102W into the high temperature sterilized air 103H produced at the outlet 118 after passing through the secondary channels 113. The second heat exchanger 112 may reach temperatures between 100° C. to 400° C. or even in excess of 1000° C. depending on applications. An insulating housing 125 including thermal insulators may be disposed to surround the second heat exchanger 112 to reduce the loss of thermal energy to the housing walls, as well as to protect the user from high-temperature surfaces.

The hot, sterilized, virus-free air 103H continues to flow through the inlet 117 of the secondary side of the first heat exchanger 110 through the secondary channels 113. The sterilized air 103H may be cooled (without mixing) by heat transfer with the colder contaminated air 102 flowing through the primary side of first heat exchanger 110. As a result, the cooled sterilized air 103 at the thermodynamic “state E” exits from the first heat exchanger 110 at the outlet 118 and, depending on the final application, it may be subjected to further cooling via heat transfer with environmental air via a third heat exchanger 124. The third heat exchanger 124 may include fins 122 thermally coupled to the sterilized air 103 on one end and to the contaminated cooler air 102 on the opposite end, so as to execute cooling functions by natural convection between the contaminated air 102 outside of the air sterilizer 100 and the sterilized air 103 flowing internally inside the air sterilizer 100. In some embodiments, the third heat exchanger 124 may execute additional cooling of the sterilized air 103 through a combination of natural and forced convection heat transfer mechanisms with the contaminated air 102 without mixing the sterilized air 103 with the contaminated air 102 external to the air sterilizer 100. The third heat exchanger 124 may include a cooling fan 123 to reset the temperature of the sterilized air 103 flowing out at the outlet 106 from the thermodynamic state F back to state A (initial state, see FIG. 2B), thus providing the sterilized air 103 at an adequate environmental temperature for inhalation by users of the air sterilizer 100.

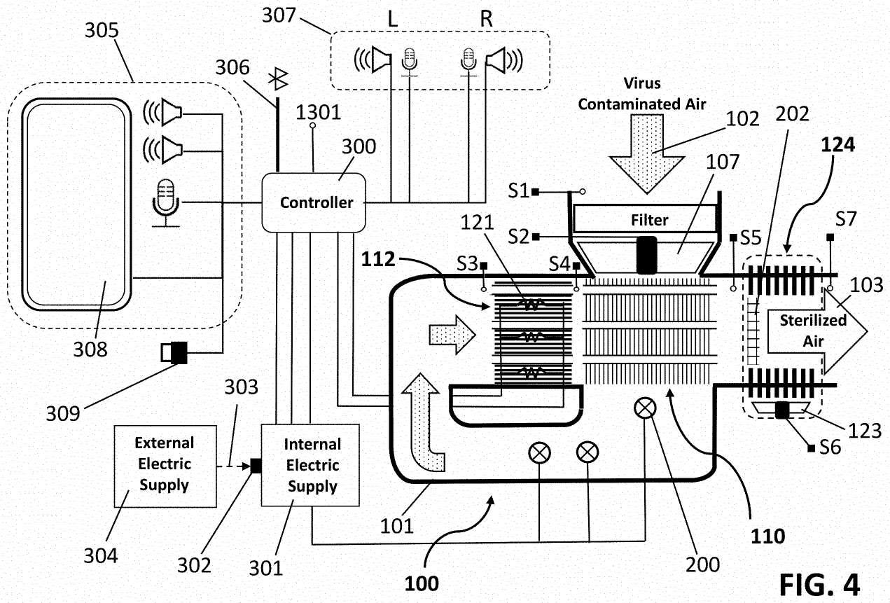

FIG. 2A shows a block diagram of the open-loop thermodynamic cycle experienced by the contaminated air 102 as it is processed while flowing through the filter 108, the first heat exchanger 110, the second heat exchanger 112, and the third heat exchanger 124. As the contaminated air 102 flows through the air sterilizer 100, the contaminated air 102 changes its thermodynamic state when superheated to a sterilizing temperature of 100° C. or higher (e.g., 100° C.-1000° C.). The temperature may be controlled by a controller 300 shown in FIG. 4.

FIG. 2B is a representative temperature plot indicating the different thermodynamic states from the initial state A to the resetting state F, as experienced by the contaminated air 102 when it flows through the various stages inside the air sterilizer 100.

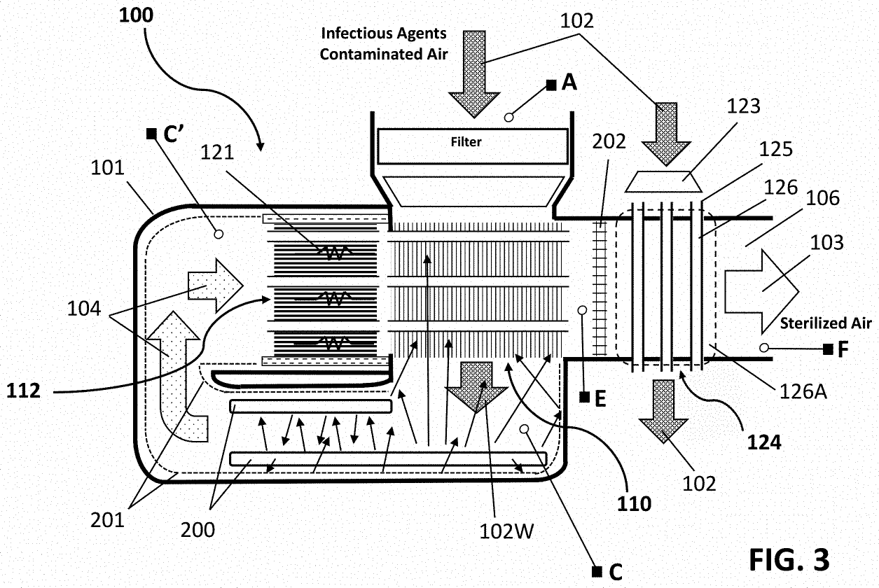

As shown in FIG. 3, the air sterilizer 100 shown in FIG. 1 may also include redundant air sterilizing features, such as the third heat exchanger 124 with increased effectiveness with a set of Ultra-Violet (UV) lamps 200 configured to operate during normal air sterilization processes and to execute sterilization of the air sterilizer 100 after use. The UV lamps 200 may also be replaced by infrared lights or heaters and provide augmentation of the air sterilization capabilities during normal operation of the air sterilizer 100. For simplicity of discussion, components that are the same or similar to those shown in FIG. 1 are not repeatedly described. As shown in FIG. 3, the UV lamps 200 may be disposed within the housing 101, and may be configured to operate at wavelengths optimized for disinfection (e.g., UV-C type) of bacteria, algae, protozoa, and viruses. The UV light emitted by the UV lamps 200 may further be distributed by one or more mirrors or mirror finished internal walls 201 of the housing 101. The mirror finished internal wall 201 may include a reflective coating to reflect the UV light. Alternatively, an adhesive film can be placed on the inner walls of housing 101 to provide the same functions as the mirror finished wall 201 on one side, and thermal insulation on its opposite side. Accordingly, one side of adhesive film 201 may be reflective (mirroring light), the other side adhered to the inner walls of housing 101 may be thermally insulating housing 101. To increase the scattering of the UV light inside the first heat exchanger 110 and throughout the internal components included in the housing 101, a light “labyrinth” may be formed. Accordingly, the internal surfaces forming the first heat exchanger 110 may be polished with a mirror finish while partially surrounding and forming structural parts of the components supporting the UV lamps 200. By configuring the internal surfaces of the air sterilizer 100 with mirror surfaces, the UV light emitted is scattered through clearances and channels, through which the contaminated air 102 flows prior to entering the second heat exchanger 112. In FIG. 3, the scattered UV light is represented by a series of arrows in various random directions. UV radiation absorption impacts the RNA and DNA forming the infectious agents including viruses, bacteria and microbes. For example, UV radiation can destroy the nucleic acid forming RNA and DNA, thus impairing or inhibiting the ability for virus particles, bacteria and microbes to replicate. UV radiation also produces toxic Ozone, which at standard environmental conditions can linger for several hours. As the UV irradiated air temperature is elevated when passing through the second heat exchanger 112, the process of ozone decay back to oxygen is accelerated as the temperature of the air-ozone mixture is increased. The UV lamps 200 can be replaced by infrared lamps/heaters to augment the air super-heating properties of the air sterilizer 100 and to avoid the need to process the Ozone by-product of UV lights operations. Prior to inhalation of the sterilized air 103 flowing from the outlet 106, the thermodynamic state F is reset to the initial thermodynamic state A (as shown in FIGS. 2A and 2B) by the third heat exchanger 124. The third heat exchanger 124 may be a PCHE or a tube-and-shell heat exchanger, in which the contaminated air 102 flows by forced convection induced by the motor-driven cooling fan 123. The cooling fan 123 may cool the contaminated air 102 flowing through the primary side of tubes 126 (or channels for PCHE types of heat exchangers). As a result, the contaminated air 102 enters the tube side of the third heat exchanger 124 at one end of the tubes 126 and exhausts at the opposite end of the tubes 126. Overall, the sterilized air 103 at the thermodynamic state E flows through the secondary side of the tubes 126 (shell-side 126A, or secondary side channels for PCHE types of heat exchangers) to be further cooled by thermal exchange, without mixing with the contaminated air 102.

In some embodiments, the UV lamps 200 may be replaced by infrared lamps or heaters. In some embodiments, the UV lamps 200 operate at a low power. In some embodiments, the UV lamps 200 may be operated intermittently. In some embodiments, the UV lamps 200 may operate in a continuous mode. Depending on the operation of the air sterilizer 100, the UV lamps 200 operate periodically to disinfect the internal components of the air sterilizer 100. In some embodiments, the UV lamps 200 may operate (or be active) during a normal operation of the air sterilizer 100. For example, the UV lamps 200 may be activated to sterilize the air sterilizer 100 after a predetermined number of hours of use. In some embodiments, the power and time exposure of the UV lamps 200 may be controlled during production (and inhalation) of the sterilized air 103. Depending on the power rating and time of activation of the UV lamps 200, Ozone (O₃) can be produced through UV irradiation reaction with the oxygen contained in the contaminated air 102. Ozone is a powerful oxidizer and contributes to neutralizing virus and other infectious agents, and is produced during operations in quantities proportional to the time the UV lamps 200 are energized at a given rated power and irradiation efficiency. These parameters may be controlled by a controller 300 shown in FIG. 4. Ozone decays in a relatively short amount of time when subjected to a high temperature. The high-temperature components of the air sterilizer 100 include the second heat exchanger 112 and the heaters 121, whose parameters may also be monitored and managed by the controller 300. Depending on the operations of the UV lamps 200, and the thermodynamic state of contaminated air 102, small amounts of Ozone may still be present at the outlet of first heat exchanger 110. The Ozone may be recombined (into neutral oxygen) prior to the sterilized air 103 exiting the outlet 106. This can be performed by a catalytic recombiner or electrostatic grid 202, which may be positioned at the inlet or be an integral part of third heat exchanger 124. As warm contaminated air 102W flows through the UV lamps 200 and the mirror and light-scattering labyrinth inside the housing 101, it changes its thermodynamic state from thermodynamic state C to a state C′, and forms the warm contaminated air 102W to a partially treated or sterilized (via high temperature exposure) air 104 (FIG. 3). In the embodiments in which the UV lamps 200 are active during the production of sterilized air 103, UV radiation adds thermal energy and further neutralizes infectious agents carried by the air 102W. The thermal loading on heaters 121 of the second heat exchanger 112 may be reduced. The UV lamps 200 and the second heat exchanger 112 represent two independent and redundant sterilizing systems each capable of neutralizing infectious agents within the contaminated air 102. Each sterilizing system is further equipped with internal redundancies (e.g., multiple heaters 121, multiple UV lamps 200) monitored and controlled by the controller 300.

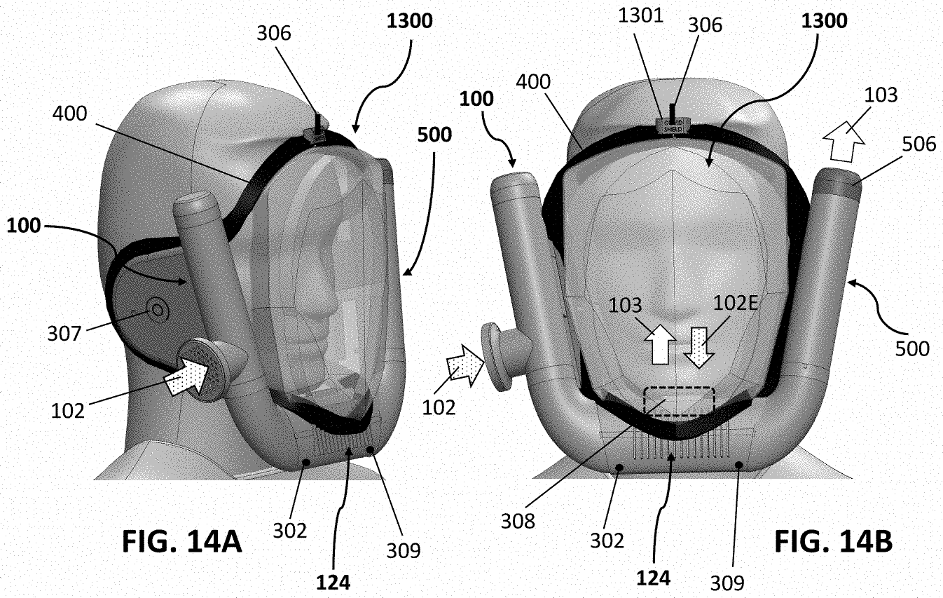

FIG. 4 shows that the air sterilizer 100 includes the controller 300, which may be an electronic controller. The controller 300 may comprise main components, sensors, actuators and hardware disposed within the housing 101. For applications of the air sterilizer 100 fully integrated with a wearable mask 1300 as shown in FIGS. 14A and 14B, the controller 300 may be equipped with microprocessors, software and electronic hardware configured to support wireless communication (e.g., Bluetooth connection) with external devices, such as smart phones and computers with apps and software dedicated to download data from the wearable mask 1300. The controller 300 may also include electronic hardware and software to support stereo audio (e.g., speakers, earphones, etc.) and microphones 307, which are configured to communicate audio information from the user wearing the wearable mask 1300 to other operators (outside of the wearable mask 1300) and to another user(s) of similar wearable mask(s) 1300. The controller 300 may be configured to process audio to provide the user of the mask 1300 spatial sound directionality perception. The controller 300 may control the microphones 307 to provide noise-suppression capabilities when the mask 1300 is used in noisy environments (e.g., hospital/ICU settings, factories, transport vehicles). The mask 1300 may be used by doctors, nurses, other hospital employees, or patients, such that these people are protected. In some embodiments, the mask 1300 may be used in the ICU rooms where the viral loads may be high. The controller 300 may be further configured to manage and support a user interface 305. The user interface 305 may include audio-microphone components and a display 308. The user interface 305 may display text, audio, and/or video information received from a smart phone or computer of the user that may be wirelessly connected with the air sterilizer 100. The user interface 305 may also receive user's voice command to operate the smart phone or computer of the user. Thus, the air sterilizer 100 may eliminate the need to “touch” the smart phone or computer, for example, to read and transmit text messages. Touching the smart phone may potentially contaminate the smart phone screen with infectious agents. The controller 300 may be further configured to manage power consumption and functional operations of the device 100. Accordingly, the display 308 may be positioned within the frontal visor of the mask 1300 as a reflected screen or as a solid screen, configured to report status of the internal power supply 301 (e.g., battery charge state), as well as text messaging information received by the user's smart phone and to convert user's audio into text message in response (e.g., through voice command). The controller 300 manages and regulates charging power of the power supply 301 and alerts the user of the status on the connection of a physical power cable 303 via a magnetic connector 302. The user alerting system is represented by video information displayed by the display 308 (e.g., reflected on the visor of the mask 1300) and audio information broadcasted by the speakers included in the device 100. The power supply 301 may include one or more battery packs with a sufficient capacity to enable the operator wearing the mask 1300 to inhale the sterilized air 103 for planned and programmable time durations, with the possibility to connect the power cable 303 to any available external electric power source, outlet, or supply 304, regardless of voltage rating (e.g., AC electric outlets and, for example, automotive DC electric power sources). The magnetic connector 302 is configured to enable inadvertent disconnection of the power cable 303 without damaging the air sterilizer 100. When the power cable 303 is disconnected during charging time, a notification of battery charging and power cable status may be displayed on the display 308, which is visible to the user. Updating the controller 300 software can be executed through a communication device 306 (e.g., a Bluetooth transceiver, a WiFi transceiver, etc.) configured to provide a wireless connection, or through a data cable 309 with a connector configured to provide a wired connection of the air sterilizer 100 with an external device outside of the mask 1300. The internal power supply 301 can be configured to include one or more battery packs and power charging-conditioning components. The external power supply 304 may be configured to also include one or more auxiliary battery packs as well as AC conditioning components.

The controller 300 monitors the thermodynamic state of the contaminated air 102 and the sterilized air 103 (e.g., states A through F described in FIG. 2B and FIG. 3) by a plurality of sensors S1-S7. These sensors may be configured to provide electronic data regarding temperature, pressure, air-flow, motor-fans speed. For example, sensors S2 and S6 may measure data relating to the motor 109 of the compressor 107 and the cooling fan 123, respectively. Temperature, pressure and air flow-rate data are sampled at various locations within the housing 101 of the air sterilizer 100. Based on data from S1-S7 sensors, the controller 300 regulates the power-speed of the compressor 107, the power and time activation of the UV lamps 200, the power-speed of the cooling fan 123, the electrical heaters 121, and the catalytic recombiner 202 while providing visual and audio notifications to the user interface 305. In some embodiments, when the air sterilizer 100 is activated, the second heat exchanger 112 including the electrical heaters 121 is actuated to superheat the air inside the air sterilizer 100 to a predetermined temperature sampled by S4, while the compressor 107 is actuated to circulate and compress the contaminated air 102 at a predetermined flow rate of at the inlet of first heat exchanger 110. Similarly, and depending on the mode of operation of the device, the UV lamps 200 are actuated, continuously or intermittently, while the controller 300 regulates the power on the electrical heaters 121 to reach a temperature at a sampling point (e.g., measured by the sensor S4) that assures air sterilization via neutralization of virus, bacteria, microbes contained in the contaminated air 102. As the sterilized air 103 flows through the third heat exchanger 124, the controller 300 regulates cooling of the sterilized air 103 by varying the speed of the cooling fan 123 based on the data measured by the sensor S7, confirming that the temperature of the sterilized air 103 is reset to the temperature reported by the sensor 51. As the filter 108 becomes clogged, the controller 300 analyzes and compares mapped information from the sensors S1-S3 and notifies through the user interface 305 that the filter 108 needs to be replaced. As a result of operation, the filter 108 may contain infectious agents. To sterilize the filter 108 and other components inside the air sterilizer 100, the controller 300 may be programmed to cycle high-temperature air by alternating the rotary direction of compressor 107 so as to increase the temperature of the filter 108 and sterilize it prior to replacing it.

The mask 1300 may be regarded as a part of the air sterilizer 100, or may be regarded as an element external to the air sterilizer 100. The mask 1300 may include an air inlet and an air outlet. The air inlet may be coupled to a sterilized air outlet of the air sterilizer 100, and may receive the sterilized air. The air outlet may be coupled to a contaminated air inlet of the air sterilizer 100, such that the air exhaled by the user can be sterilized by the air sterilizer 100.

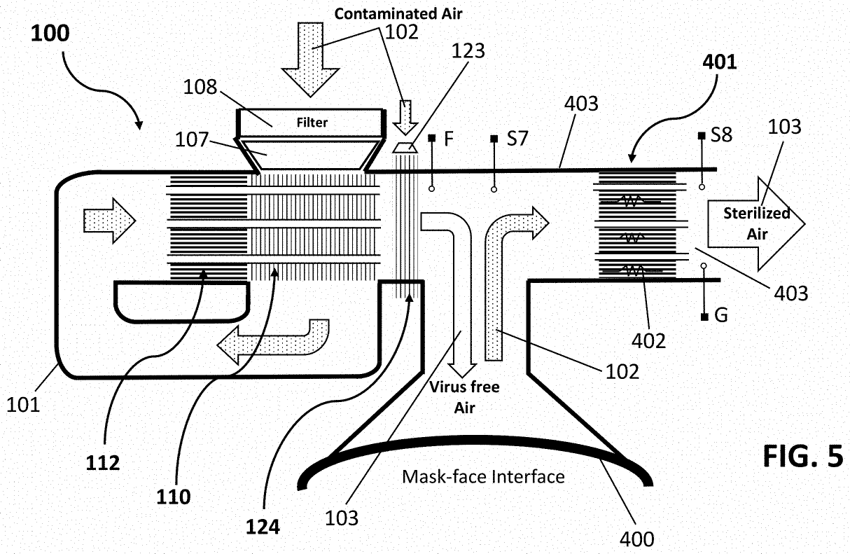

FIG. 5 is a schematic illustration of the air sterilizer 100 with the addition of a mask-face interface 400 configured to hermetically seal the user's face including the areas surrounding the user's ears. In some embodiments, the air sterilizer 100 includes a fourth heat exchanger 401 configured to sterilize the user's exhaled air (potentially contaminated by the user) prior to venting to atmosphere at an exhaust outlet 403. The fourth heat exchanger 401 may include electrical heaters 402 controlled by the controller 300. The pressure state at the sensor S7 enables the controller 300 to regulate the compressor 107 and the cooling fan 123 to produce the sterilized virus-free air 103 proportionally to a user's inhalation dynamic (time-dependent) demand. As the user may be infected, for example, with corona virus, the exhaled air may be contributing to spreading airborne infectious particles and further contaminate the contaminated air 102. The fourth heat exchanger 401 may increase the temperature of the potentially contaminated air 102 exhaled by the user.

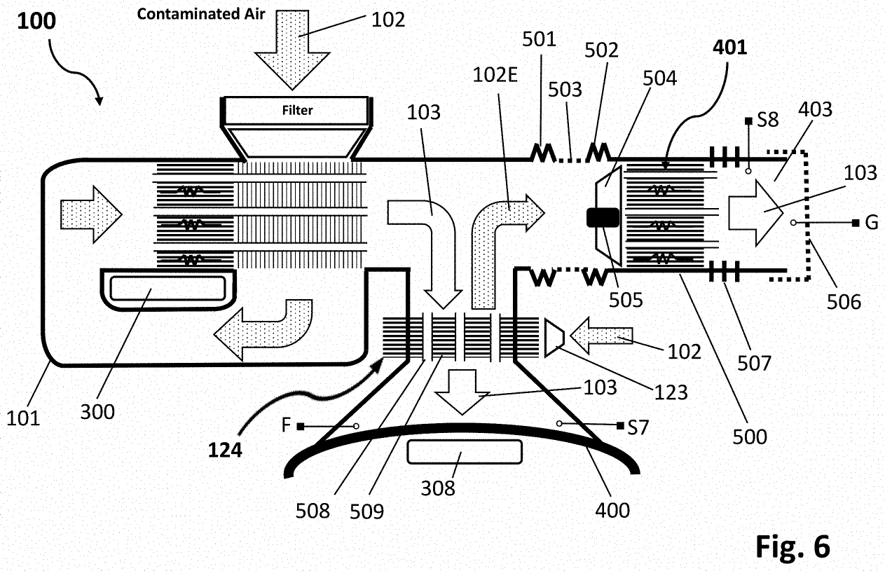

FIG. 6 illustrates the air sterilizer 100 with the fourth heat exchanger 401 configured to superheat an exhaled contaminated air 102E and an auxiliary compressor 504 driven by a motor 505 to compress the exhaled contaminated air 102E into the fourth heat exchanger 401. The air sterilizer 100 may also include a flexible coupler 502 configured to seal and hydraulically couple an auxiliary assembly 500 to a flexible-long or short-rigid tubing 503. The air sterilizer 100 may also include a flexible coupling 501 configured to seal and hydraulically couple the flexible-long or short-rigid tubing 503 to the housing 101. The air sterilizer 100 may also include a heat shield exhaust screen 506 configured to exhaust the sterilized air 103 to the environment. To increase the cooling effectiveness of the third heat exchanger 124, the surfaces of finned portions 509 are extended and thermally coupled to channels 508 that exchange thermal energy with the sterilized air 103 to adjust the temperature of the sterilized air 103 for user's inhalation by regulating the speed of the cooling fan 123 (e.g., via controller 300 shown in FIG. 4). The sensor S8 coupled to the controller 300 provides thermodynamic comparative data along with data from the sensor S7 to regulate the cooling fan 123 and the speed of the auxiliary compressor 504 to adjust to the user breathing rate. General status information of the air sterilizer 100 is provided through processing data by the controller 300 transmitted through the display 308 integrated with the visor of the mask 1300 (FIGS. 14A-17). As the virus-free air 103 exits the fourth heat exchanger 401 at the exhaust outlet 403, its thermodynamic state G is characterized by a relatively high temperature. This temperature is lowered by natural convection with cooling fins 507. The cooling fins 507 may be configured to transfer thermal energy from the hot virus-free air 103 at one end of the cooling fins 507 inside the housing of the auxiliary assembly 500 at the outlet 403 to the environmental air at the opposite end of the cooling fins 507 outside of the housing of auxiliary assembly 500.

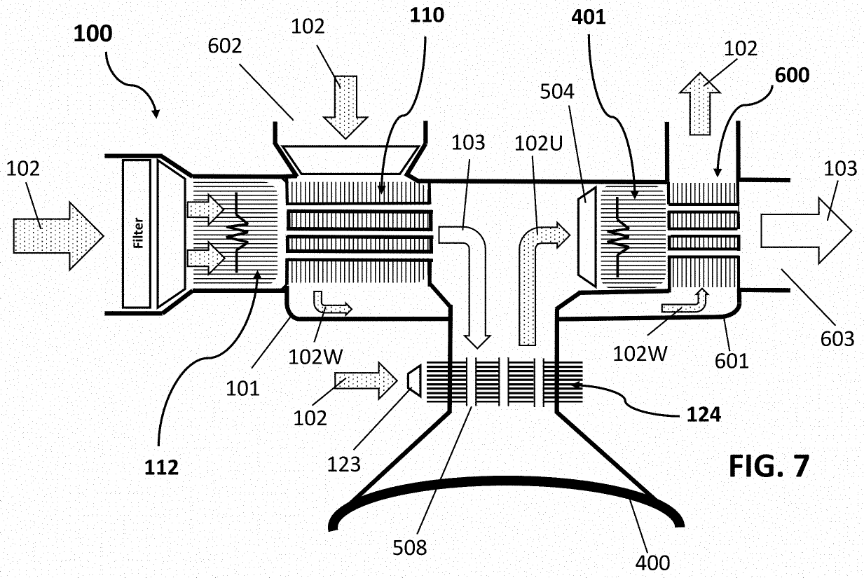

FIG. 7 shows the air sterilizer 100 with the housing 101 and a housing 601 so as to form an internal open loop for the contaminated air 102 to provide cooling to the first heat exchanger 110 and a fifth cooling heat exchanger 600. The contaminated air 102 enters the first heat exchanger 110 at an inlet 602, cools down the superheated sterilized air 103 produced in the second heat exchanger 112 and flows within an internal conduit formed by the housing 101 and the housing 601 so that the warm contaminated air 102W flows through the fifth heat exchanger 600 to cool down the sterilized air 103 at an outlet 603. One of the differences between the configurations shown in FIGS. 6 and 7 is that the cooling fins 507 (which uses natural convection for heat exchange) are replaced by the fifth heat exchanger 600 (which uses forced convection for heat exchange). The remaining components included in the air sterilizer 100 shown in FIGS. 6 and 7 are similar.

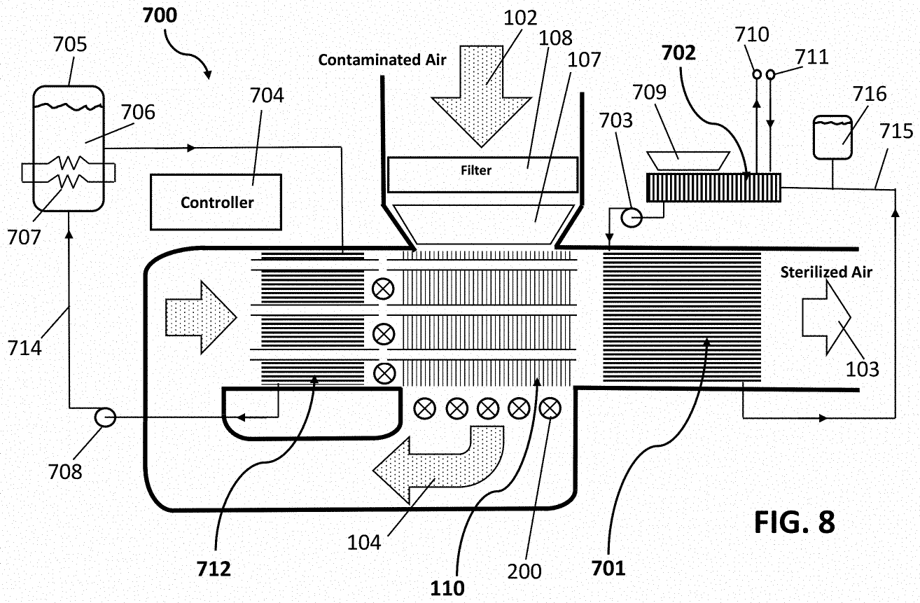

FIG. 8 shows the air sterilizer 100 configured to sterilize larger volumes of air. This embodiment of the air sterilizer 100 is referred to as an air sterilizer 700. The air sterilizer 700 is scaled proportionally to the amount of the contaminated air 102 to be sterilized. The functioning principles and components forming the air sterilizer 700 are similar to those described in previous figures. The air sterilizer 700 may include scaled-up components and high-effectiveness heat exchangers. The high-effectiveness heat exchangers may utilize a first working fluid 706 for the transfer of high-temperature thermal energy to the air to be treated and a second working fluid 716 for the cooling of the sterilized virus-free air 103 prior to discharging into the controlled environments as, for example, ICU room, ER, hospital rooms, factories, indoor facilities, grocery stores apartments and homes as well as high-occupancy vehicles as those utilized for public and military transportation. The infectious agents and/or virus contaminated air 102 enters the air sterilizer 700 through the scalable particulate filter 108 by means of the scalable compressor 107. The air sterilizer 700 includes a proportionally scaled first heat exchanger 110 with functioning principles as described for FIGS. 1-7. In the configuration shown in FIG. 8, the first working fluid 706, heated by a heat source, for example, controlled heaters 707, is circulated through a closed loop 714 by means of a recirculator pump 708. The first working fluid 706 may be any suitable thermodynamic fluid that may function at a high temperature as, for example, thermal-oil. The first working fluid 706 may be heated in a controlled manner by energizing the heaters 707 controlled by a controller 704 with thermodynamic states control as described in connection with FIG. 4. As hot first working fluid 706 circulates through a second heat exchanger 712, the pre-treated air 104 is superheated. The pre-treated air 104 and first working fluid 706 are thermally coupled but are not physically mixing. As the pre-treated air 104 potentially carrying infectious agents is superheated when flowing through the second heat exchanger 712, it flows through the first heat exchanger 110 to be cooled down as the first heat exchanger 110 operates as a cooler and a recuperator heat exchanger at the same time. The second working fluid 716 circulates through the third heat exchanger 701 thermally coupled to the sterilized air 103 to cool down the sterilized air 103 before it is discharged into a controlled environment. The second working fluid 716 may be any working fluid with suitable thermal-physical properties, including thermal-oil, engineered organic fluids, even water. The second working fluid 716 circulates by means of a pump-circulator 703 while rejecting thermal energy to the environment through a radiator 702. The radiator 702 may be air-cooled by actuating and regulating a fan 709. The radiator 702 may also be water cooled by circulating a fluid (e.g., water) through conduits 710 and 711. The effectiveness and heat load rating of the heat exchangers included in the air sterilizer 700 depend on the type of heat exchangers (e.g., PCHE, tube-and-shell, others), and on sizes selected proportional to the volumes of indoor contaminated air to be treated.

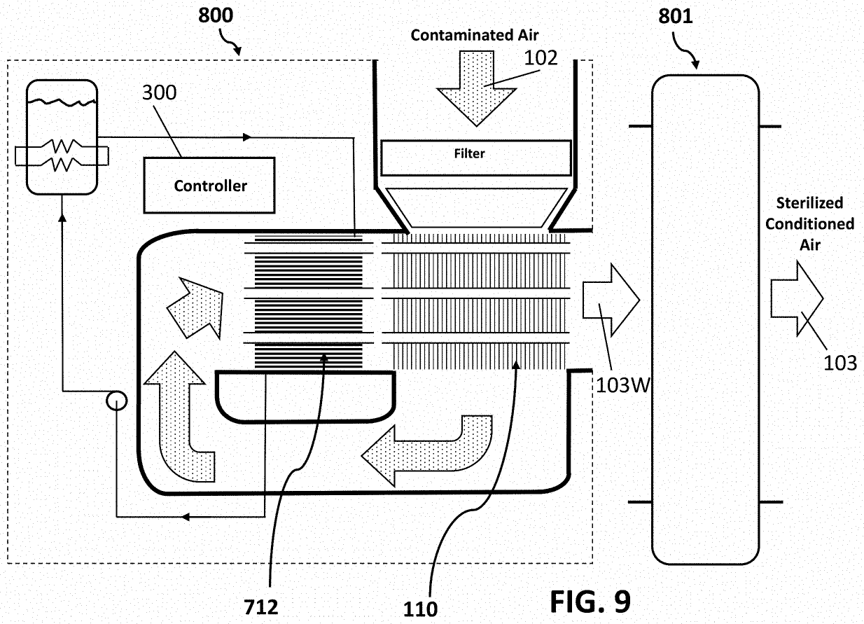

FIG. 9 shows an embodiment of the air sterilizer 100 or 700 described above in connection with FIGS. 1-8. For convenience, the embodiment shown in FIG. 9 is referred to as an air sterilizer 800. The air sterilizer 800 is configured to treat large volumes of virus-contaminated air as for the air sterilizer 700 shown in FIG. 8. The air sterilizer 800 may further include an auxiliary air conditioning system 801 to cool the sterilized air 103W and lower its temperature prior to venting it within a controlled volume (e.g., ICU rooms, ER, hospital's rooms, factories, indoor offices, grocery stores, restaurants, homes and cabins of transportation vehicles). The working principles and components included in the air sterilizer 800 are similar to those described above in connection with FIGS. 1-8. The auxiliary air-conditioning system 801 may be any suitable air-conditioning unit configured to provide indoor heated or cooled air.

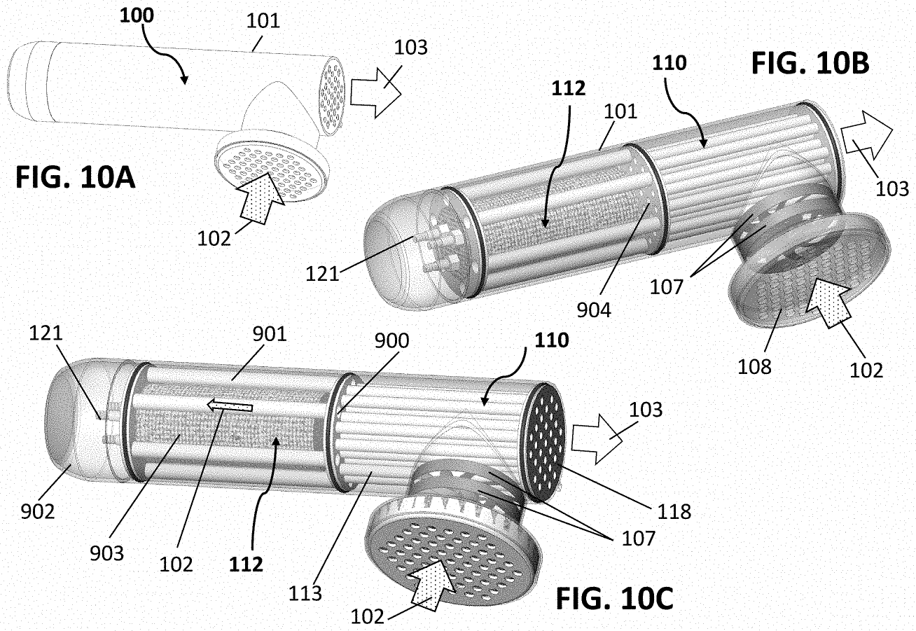

FIGS. 10A-10C illustrate perspective and transparent views of a configuration of the air sterilizer 100 described above in connection with FIGS. 1-7. FIG. 10A shows a simplified configuration of the air sterilizer 100 made with simple geometries and tube-and-shell heat exchangers. The second channels 113 of the first heat exchanger 110 may be made by tubes in a cross-flow configuration with respect to the direction of the flow of the infectious agents contaminated air 102. The contaminated air 102 may be compressed inside the housing 101 by the compressor 107, which may be a counter-rotating double stage axial compressor, after passing through the filter 108. As the contaminated air 102 flows through the first heat exchanger 110, it cools down the sterilized air 103 which exits the air sterilizer 100 at the outlet 118. As the contaminated air 102 flows through the primary side (shell-side) of the first heat exchanger 110, it heats up and enters the second heat exchanger 112 through channels 901 via inlets 900 as shown in FIG. 10C to accumulate within a chamber 902. In the chamber 902, the warm contaminated air 102 executes flow inversion and enters the second heat exchanger 112 through a porous metal component or a PCHE 903 configured to transfer thermal energy from the electrical heaters 121, thermally coupled to second heat exchanger 112 to the contaminated air 102. As the contaminated air 102 is superheated through the second heat exchanger 112, it flows through the tube side 904 (FIG. 10B) of the first heat exchanger 110 to cool down through thermal exchange with the cooler contaminated air 102 flowing on the shell-side of the heat exchanger to produce sterilized cooled air 103 at the outlet 118.

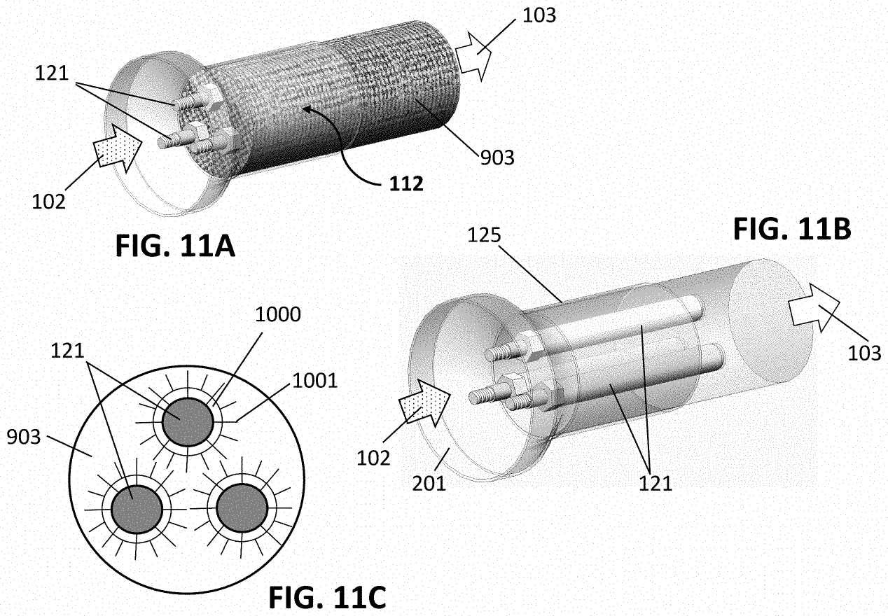

FIGS. 11A-11C illustrate perspective, semi-transparent and cross-sectional views of the heating cartridge formed by the second heat exchanger 112. In this configuration, the second heat exchanger 112 includes the electrical heaters 121 embedded with and thermally coupled to a porous thermally conductive material or a PCHE 903 to extend the heated surfaces of the second heat exchanger 112 to effectively superheat the contaminated air 102 flowing through clearances 1000 and fins 1001 thermally coupling the electrical heaters 121 to the channels or porous material of the PCHE 903. To minimize thermal energy losses outside of the air sterilizer 100, the second heat exchanger 112 is housed within the insulating housing 125 with the mirror finished internal walls or mirrors 201 and insulating liner to reflect UV radiation for the configurations utilizing UV lamps 200 or infrared radiation if equipped with infrared heaters as described in connection with FIG. 3.

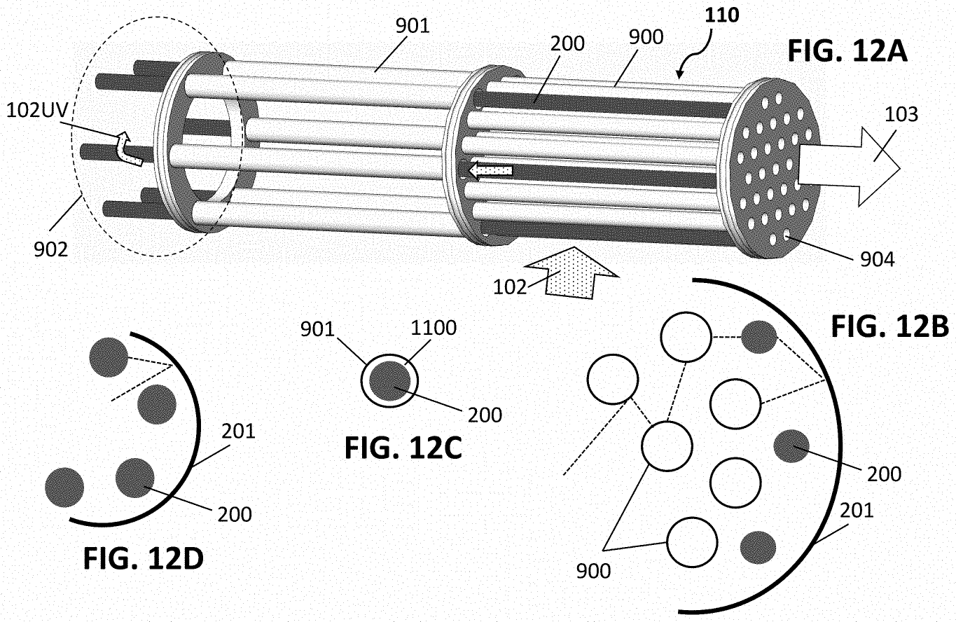

FIGS. 12A-12D illustrate perspective and semi-cross-sectional views of an internal configuration of the air sterilizer 100. The UV lamps 200 are positioned within the first heat exchanger 110 and extend within the channels 901 so as to form a clearance 1100 (FIG. 12C). The contaminated air 102 flows through at a high exposure to UV radiation prior to exiting into the chamber 902 (shown in FIGS. 10A-10C). In this manner, the contaminated air 102 is irradiated and disinfected to become sterilized air 102UV prior to undergoing superheating processes through the second heat exchanger 112 as shown in FIGS. 10A-10C. To increase UV irradiation, the external walls of the inlets 900 of the first heat exchanger 110 are mirror-finished or coated with a reflective film to execute functions similar to the mirror finished internal walls 201 of the housing, so as to generate scattered UV light throughout the interior space of the device 100. In some configurations, UV lamps 200 may be replaced by infrared heating elements.

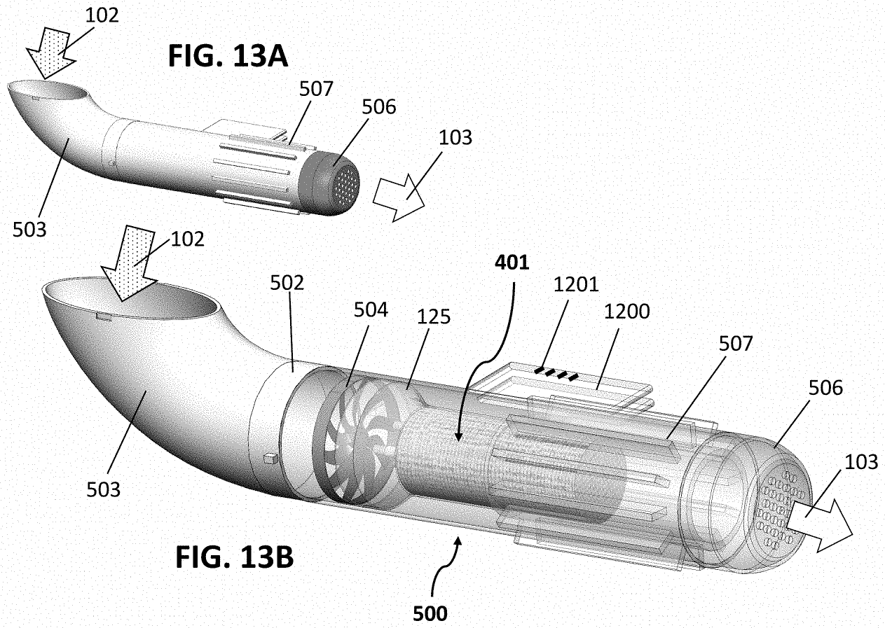

FIGS. 13A and 13B show a perspective view of the auxiliary assembly 500. In some embodiments, the auxiliary assembly 500 may be a superheating cartridge 500. The superheating cartridge 500 may be configured to sterilize potentially contaminated air 102 exhaled by an infected user. The superheating cartridge 500 may be configured to enable flow and exposure of the infectious agents and/or virus-contaminated air 102 to extended high-temperature surfaces formed by channels of a PCHE or a thermally conductive porous material of the fourth heat exchanger 401. The fourth heat exchanger 401 may be thermally coupled to the cooling fins 507, which is thermally coupled with the environmental air outside of the superheating cartridge 500, to cool down the sterilized superheated air at the outlet of the fourth heat exchanger 401. A mechanical support 1200 may be configured to be assembled with the mask 1300 shown in FIGS. 14A and 14B. The mechanical support 1200 may include electrical connections for the control and actuation of the electrical heaters within the fourth heat exchanger 401 while providing sensor data communication with the controller 300.

FIGS. 14A and 14B illustrate a configuration of the air sterilizer 100 and the superheated cartridge 500 integrated with the mask 1300 equipped with the mask-face interface 400. The wearable mask 1300 enables sterilization of the contaminated air 102 prior to inhalation and prior to venting exhaled air 102E back to the environment. The wearable mask 1300 includes one or more of the features shown in FIGS. 1-7, 10A-13B. The wearable mask 1300 may further include data visualization through the display 308, which may inform the users about whether the user has fever through a sensor 1301 configured to sample a skin temperature. As the controller 300 may be paired to the user's smart phone with GPS capabilities, information on user's fever status may also be transferred to a data center to provide quarantine notification and location for the user who developed fever while wearing the mask 1300.

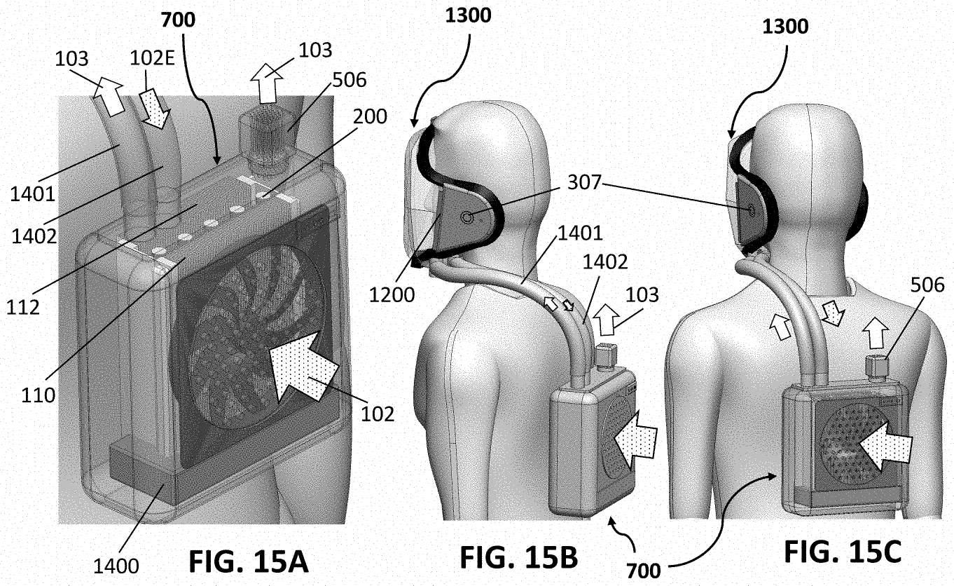

FIGS. 15A-15C illustrate a “backpack” configuration of the air sterilizer 700. In this configuration, a larger battery pack 1400 (similar to the internal electric power supply 301 shown in FIG. 4) may be included in the air sterilizer 700 configured to process a larger amount of contaminated air 102 for prolonged period of time. In this configuration, the air sterilizer 700 may include flexible conduits 1401 and 1402 dedicated to hydraulic connection of the virus-free air 103 to the wearable mask 1300, and hydraulic connection of exhaled contaminated air exhaled 102E for treatment and exhaust as sterilized air 103. As shown, the wearable mask 1300 may be hydraulically connected via the flexible conduits 1401 and 1402 for inhalation of the sterilized air 103, and for treatment of the exhaled air 102E to the air sterilizer 700 for further processing of the exhaled air prior to venting it to the environment.

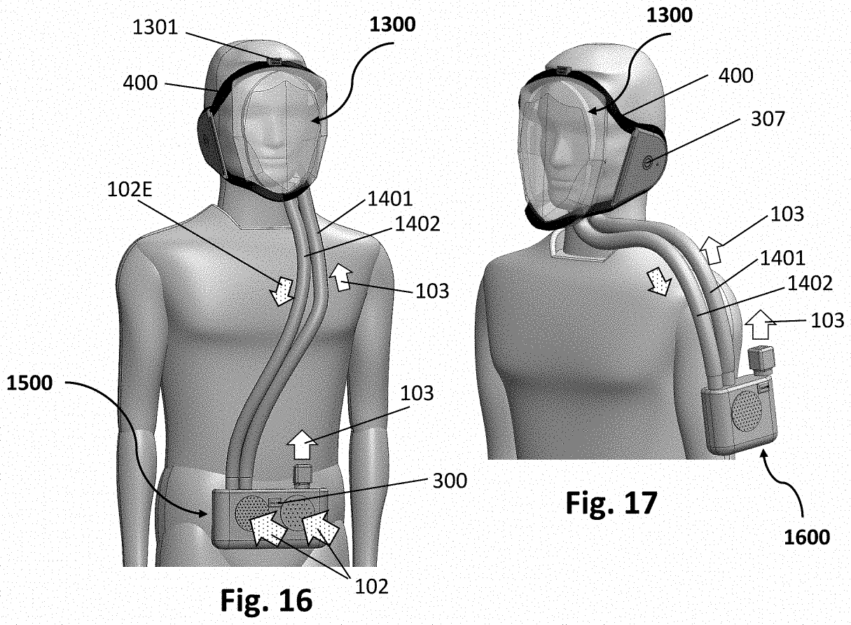

FIG. 16 illustrates a wearable belt-unit 1500 configured to house the air sterilizer 100 or 700. The belt-unit 1500 may include flexible conduit(s) coupling the belt-unit 1500 to the wearable mask 1300.

FIG. 17 illustrates a wearable arm-unit 1600 configured to house the air sterilizer 100 or 700. The flexible conduits 1401 and 1402 may couple the arm-unit 1600 to the wearable mask 1300.

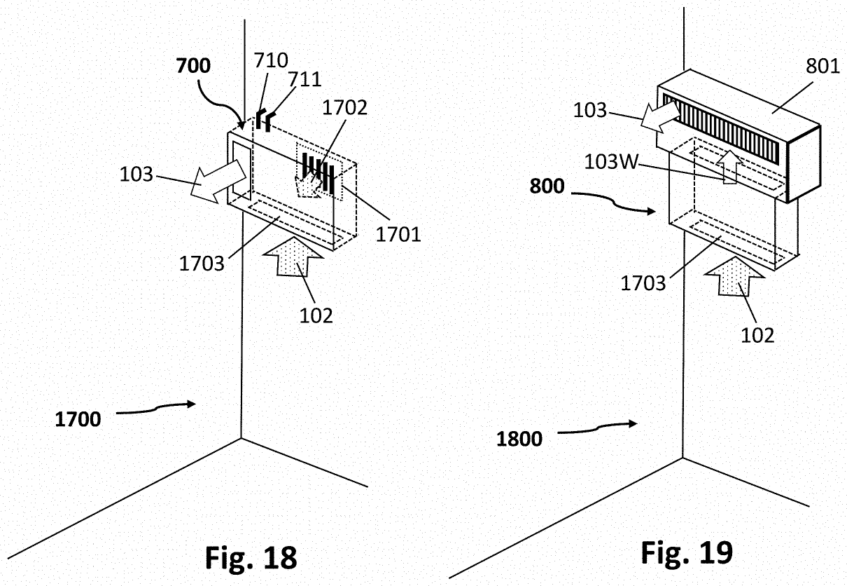

FIG. 18 illustrates a configuration of the air sterilizer 700 for applications requiring indoor processing of large volumes of infectious agents and/or virus-contaminated air 102. The configuration may or may not include an integrated water-cooling system with the water circulation conduits 710 and 711 for conditioning the air prior to venting it indoor at a predetermined temperature. In this embodiment, the air sterilizer 700 may be configured for applications within an indoor environment 1700 (e.g., ER, ICU room, hospital rooms, grocery stores, factories, offices, homes as well as vehicles cabins). The air sterilizer 700 may be mounted to an air-conditioned outlet 1701 at the wall, floor, or ceiling. The contaminated air 102 from the indoor environment 1700 and/or from the centralized air conditioning system networked to the air-conditioned outlet 1701 is circulated within the air sterilizer 700. Accordingly, the contaminated air 102 in the indoor environment 1700 and/or a conditioned contaminated air 1702 from the air-conditioned outlet 1701 may flow first within the internals of the air sterilizer 700 to undergo superheating and sterilization as described above prior to venting as the sterilized air 103 conditioned by air-cooling through regulation of the temperature of the contaminated air 1702 or via water-cooling through the radiator 702 shown in FIG. 8, or a combination of air-cooling (e.g., circulated by the fan 709) and water cooling (e.g., circulated through the water conduits 710 and 711).

FIG. 19 illustrates the air sterilizer 800 shown in FIG. 9 and FIG. 18, for applications requiring processing of large indoor infectious agents virus-contaminated air volumes. In this configuration, the contaminated air 102 from an indoor environment 1800 is processed by the air sterilizer 800 and vented as the warm sterilized air 103W for temperature conditioning executed by typical air-conditioning and the auxiliary air-conditioning system 801 (such as heat-pump wall radiators) so as to regulate the temperature of the sterilized air 103 by circulating the air through an air-conditioning split unit normally coupled to HVAC centralized units.

Passenger railcars carry millions of passengers and represent air-conditioned indoor environments, such as hospitals, factories, schools, offices and homes, in which people are exposed to a mixture of outdoor and recirculated air. Passenger railcars represent high occupant density and confinement of occupants for prolonged periods of time. During operations, passengers intake air undergoing a combination of environmental factors including humidity and exposure to contaminants, such as carbon monoxide (CO) and organic chemicals, including virus, bacteria, micro-organisms, fungi, spores and infectious biological agents. For convenience, passenger railcars are referred to as hereafter “railcars.” The railcars operate in an environment that varies in temperature, pressure, relative humidity and contaminants. To transport passengers through different environmental conditions, a railcar may be equipped with an Environmental Control Systems (ECS) designed to maintain safe and comfortable environments for passengers and crew. The ECS is a part of the railcar equipment. The breathable air provided to passengers on railcars is a mixture of air from outside of the railcar brought into the railcar internal compartments through fans or compressors included in the railcar air-conditioning system for heating and cooling of the railcar interior. Generally, a certain amount of air is filtered as it is recirculated through the railcar interior. The ECS is configured to minimize the introduction of contaminants into the railcar interior, and to control ventilation, temperature and humidity. Most ECSs consider a minimum of 0.55 lb of external air per minute per occupant (14 CFR § 25.831). However, this ventilation flow rate is substantially lower than the ventilation flow rate recommended by the American Society of Heating, Refrigerating and Air Conditioning Engineers (ASHRAE) Standard 62-1999, developed for indoor environments. Additionally, the primary purpose of the ECS is not to eliminate contaminants, but rather to reduce the contaminant concentration by bleeding a portion of air inside the railcar and replacing it with outside air as the outside air is assumed to be contaminants free. Contaminants may include odors and gases emitted by occupants, ozone (O₃) entering the cabin from traction motors and high-power electronics often operating under the railcar or in dedicated equipment compartments, organic compounds emitted from cleaning and other materials. For example, from undercarriage equipment lubricating oils, brakes, exhaust gases for railcars powered by on-board diesel engines, particulate, hydraulic fluids. Contaminants may also include infectious agents such as viruses, bacteria, micro-organisms, fungal spores and bio-effluents. As for all indoor environments, contaminants are further exacerbated by spores possibly growing on the HVAC air-conditioning equipment and spreading through the railcar air recirculation systems (part of the ECS). Therefore, infectious-disease agents can be transmitted between railcar occupants in close proximity and distributed throughout the railcar interiors through internal recirculation of air. Overall indoor air recirculation systems increase passengers' risk of exposure to infectious-diseases agents (e.g., leading to COVID19, SARS and other diseases).

In the railcar application of the air sterilizer disclosed herein, virus containing air exhaled by infected passengers traveling relatively large distances from the source (e.g., passenger coughing or sneezing without a mask), are intercepted by “sterilized-air-curtains.” This redirects the contaminated air to air-suction ports to be collected and treated by the superheating and cooling apparatus and method of the present disclosure. The air sterilizer therefore neutralizes infectious agents prior to recirculating sterilized air back through the railcar interiors by the ECS and/or HVAC systems.

In this railcar application of the air sterilizer, the thermal energy sources may be the waste thermal energy emitted by components normally operating in a passenger railcar. The high temperature components may include, for example, ECS heat exchangers, traction motors cooling system, electronic components cooling system, brake systems, dynamic brakes and other railcar equipment whose operation generates thermal energy that is typically rejected to the environment. In some embodiments, infectious agents, virus, bacteria and germs can be neutralized by treating air circulating within the railcar interior with ozone from Ultra Violet C light (UVC) lamps generating UVC radiation and by then converting the ozone generated by UVC to neutral oxygen through catalytic converters, and/or by superheating the treated air to accelerate natural decay-conversion of ozone into oxygen by exposing ozone rich air to the high temperature components. In some embodiments, the thermal energy to heat up contaminated air can be sourced in the heat rejected to the environment by the heat exchangers of the HVAC and AC air conditioning system. The HVAC and AC equipment is part of the ECS. Therefore, high-temperature heat sources in a passenger railcar may be represented by the condenser and receiver equipping the AC system. The condenser is a heat exchanger generally configured to air-cool the refrigerant compressed by the compressor so that the refrigerant cools down while the air flowing through the heat exchanger becomes heated. The heated air can be used to increase the temperature of the contaminated air. The air sterilizer may include heaters and heat exchangers coupled to waste heat generating components to further increase the temperature of the contaminated air with minimum expenditure of electrical energy. In other words, sources of high temperature in the context of transportation vehicles such as a passenger railcar may be represented by ECS equipment executing electrical heating and by retrofitting and augmenting the HVAC air-cooled equipment (e.g. condenser) with additional heaters for the purposes of superheating the air to neutralize infectious agents. To summarize, the air superheating heat sources are utilized to increase the temperature of infectious agents contaminated air. The temperature of the contaminated air can be increased by circulating it within a heat exchanger that thermally couples it to the HVAC or AC condenser (air-conditioner or heat pump heat exchanger) to extract thermal energy from this equipment, while further heating the air with additional heaters, or via thermal energy extracted from dynamic brakes, brake system or internal combustion engine's exhaust gases for self-power passengers railcars equipped with combustion engines. Another heat source that can be utilized to increase the temperature of the contaminated air may be represented by battery packs whose charging and discharging operations causes their temperature to increase and that may need active cooling during operation. As the air is heated and infectious agents are neutralized, the treated air (sterilized air) may be cooled down through thermal coupling with environmental air (e.g., via air-cooled radiators) and recirculated back to the railcar cabin.

In some embodiments, the sterilized air may be thermally coupled to the primary side of a high-effectiveness heat exchanger (e.g., Printed Circuit Heat Exchanger—PCHE, compact fins heat exchanger, shell-and-tube heat exchangers), with the secondary side thermally coupled to high-temperature exhaust gases from the railcar equipment generating thermal energy, dedicated heaters.

Air superheating can also be obtained through electric radiative, conductive and convective heaters, infrared heaters, laser and electro-magnetic (microwave) heaters and ultraviolet radiation (UVC). These methodologies can be applied to neutralize infectious agents (e.g. leading to COVID-19 and its mutations) and effectively quasi-instantaneously sterilize the air prior to recirculating it back to the railcar cabin.

In some embodiments, the air sterilizer may be scaled proportionally to the amount of air to sterilize within a closed (indoor) environment with the possibility for the air sterilizer to supply sterilized air to multiple closed environments as those represented by compartments thermal-hydraulically coupled by HVAC and ECS ducts, inlet and outlet air vents. In some embodiments, the air sterilizer can be further scaled-up to treat viruses, harmful bacteria and micro-organisms contaminating indoor air for applications in closed environments with use of air-conditioning, as virus transmission can largely occur in well-air-conditioned environments such as hospitals, hotels, offices, factories, schools.

The present disclosure provides a scalable, electrically powered, battery-powered, or waste heat energy powered real-time air sterilizer dedicated to the sterilization of air from infectious agents. The air sterilizer may include a light-weight superheater and a cooler to enable each individual (e.g., passengers or crew) to breathe sterilized air when confined in closed, indoor, environments. The air sterilizer can be scaled and configured to take advantage of waste heat energy sources to lower the electrical consumption of the mechanisms utilized by the invention to superheat the air, and treat proportionally larger volumes of potentially contaminated air inside hospitals and other indoor facilities (hotels, factories, schools, restaurants, small- and large-shops, vehicles cabins) to mitigate and neutralize the spreading of infectious agents (virus carrying particles) transport via droplets aerosols inside closed environments.

In some embodiments, the air sterilizer may be configured to circulate virus-contaminated air through an air-energy increasing mechanism configured to increase the energy content of controlled amounts of air per unit time so as to elevate the air temperature to kill coronaviruses and any other infectious agent before venting the resulting sterilized air into the indoor environment.

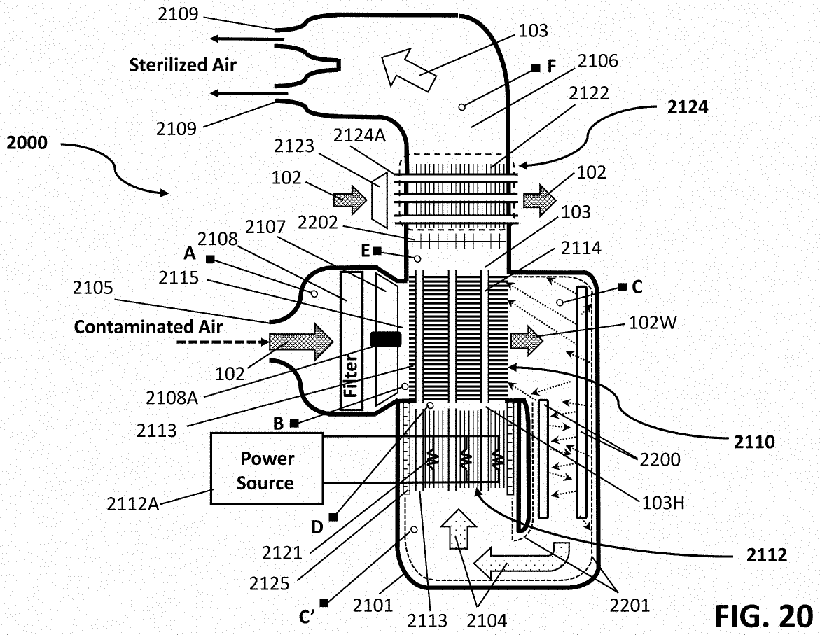

FIG. 20 shows a real-time air sterilizer 2000. The air sterilizer 2000 is based on the air sterilizer 100 described in FIGS. 1-9 and uses similar components definitions and functions. The air sterilizer 2000 may include a housing 2101 configured with at least one inlet 2105 and one or multiple outlets 2109. The contaminated air 102 flows from the inlet 2105 to the outlets 2109 by means of a compressor or fan 2107, and may be superheated by heat energy. In some embodiments, the contaminated air 102 is heated while flowing through a first heat exchanger 2110 (e.g., a recuperator heat exchanger) which increases its thermodynamic state from state A to state C (FIG. 2B). Contaminated air 102 increases its temperature as it flows through the recuperator heat exchanger 2110 and becomes contaminated heated air 102W. In some configurations of air sterilizer 2000, contaminated air 102W may undergo UV irradiation prior to undergoing superheating. Air exposed to UV radiation generates toxic ozone, however, after UV radiation exposure, the hot air 102W is superheated which accelerates the decay of Ozone process back to oxygen (ozone conversion to oxygen process).

The contaminated air 102 may be driven to the inlet 2105 by the compressor 2107, or it is compressed into the air sterilizer 2000 by external device (e.g., fans, compressors included in air-conditioning and environmental control systems). The compressor 2107 may be configured to generate a lower pressure at the inlet side of a filter 2108. Particulate and other non-gaseous contaminants contained in the air are trapped by the filter 2108. The filter 2108 may be formed by materials similar to those forming the filter 108. The compressor 2107 may be formed by a single or a multistage axial compressor or centrifugal radial turbine compressor driven, for example, by a brushless motor 2108A. The compressor 2107 may also be configured as a positive-displacement mechanical compressor driven by an electric motor (e.g. 2108A). The compressor 2107 may increase the internal pressure of the air sterilizer 2000 to overcome backpressure caused by heat exchangers, fins, channels and clearances configured within the air sterilizer 2000. The heat exchangers, fins, channels and clearances included in the air sterilizer 2000 are similar in principle to those included in the air sterilizer 100 with different scale to process generally larger volumes of potentially infectious agents contaminated air. For all applications of air sterilizer 2000 and 100, these components are configured to increase residence time and air exposure to heated surfaces as the infectious agents contaminated air (102) flows within these components. The environmental air recirculated within indoor spaces may be subject to a series of thermodynamic changes prior to being inhaled by one or multiple individuals. As described in FIGS. 2A-2B, the contaminated air 102 is at a thermodynamic state defined by an environmental temperature and a pressure denoted as a thermodynamic “state A” prior to being compressed by the compressor 2107. The contaminated air 102, after compression by the compressor 2107, is at a thermodynamic “state B”, and flows at an inlet 2115 of the recuperator heat exchanger 2110. As contaminated air 102 flows through the first heat exchanger 2110, it is thermally coupled to the air being processed by a second heat exchanger 2112 containing a superheating electrical cartridge. In some embodiments, the second heat exchanger 2112 may be coupled to an auxiliary working fluid transporting a high temperature working fluid from a high-temperature heat source. The contaminated air 102 and the sterilized air 103 do not mix. The first heat exchanger 2110 may be formed by a printed circuit heat exchanger (PCHE), configured with primary channels 2114 and secondary channels 2113. The contaminated air 102 flows at the inlet 2115 of the first heat exchanger 2110. After thermal energy exchange with the superheated sterilized hot air 103H, the contaminated air 102 flows through the primary side of first heat exchanger 2110. As the contaminated air 102 flows through the outlet of the first heat exchanger 2110, its thermodynamic state changes to “state C” (FIG. 2B) as its temperature is increased due to heat transfer with superheated air 103H flowing through the primary side of the first heat exchanger 2110.

In some embodiments, the first heat exchanger 2110 may be formed by a tube-and-shell type heat exchanger. When a tube-and-shell type of heat exchanger is utilized, the contaminated air 102 may flow through a “shell side” of the heat exchanger corresponding to the secondary channels 2113, while the superheated air 103H flows through tubes equivalent to the primary channels 2114. In some embodiments, the first heat exchanger 2110 may be formed by a compact-finned heat exchanger. The first heat exchanger 2110 may lower the temperature of the superheated air 103H from thermodynamic “state D” to thermodynamic “state E” while increasing the temperature of the contaminated air 102 from thermodynamic state B to C (FIG. 2B) in preparation for superheating through the second heat exchanger 2112. In other words, as the contaminated air 102 flows through the first heat exchanger 2110, its thermodynamic state changes from state B to state C as the energy content of colder contaminated air 102 is increased from the inlet 2115 to warm contaminated air 102W as a result of heat transfer from the hotter sterilized air 103H exiting the second heat exchanger 2112. The contaminated warm air 102W at thermodynamic state C after exiting the first heat exchanger 2110 flows through the secondary channels 2113, comprised by the housing 2101, and continues to flow through the second heat exchanger 2112, by flowing through the heating secondary channels 2113 (tubes or PCHE channels), and flows through the outlet as the sterilized hot air 103H at thermodynamic state D. The second heat exchanger 2112 can be formed by a finned, tube or PCHE heat exchanger thermally coupled to heating elements (e.g., electric heaters) 2121. The second heat exchanger 2112 may elevate the temperature of the contaminated air 102W and 104 to values that sterilize infectious agents as the air flows through the air sterilizer 2000. In some embodiments, warm air 102W is subjected to UVC lights 2200 as it travels within the internal conduits of air sterilizer 2000. As a result of UVC exposure, the air 2104 may be contaminated with toxic ozone. The decay of toxic ozone back to neutral oxygen may be accelerated by superheating the air by means of thermal energy transfer, e.g., via the electric heaters 2121, resulting into superheated air 103H. To superheat the air from thermodynamic state C′ to D (FIG. 2B), the electric heaters 2121 can be configured to transfer thermal energy into the second heat exchanger 2112. The electric heaters 2121 may be formed, for example, by independent clustered resistive elements (e.g., glow plug types), printed circuit or infrared heaters. The electric heaters 2121 transfers thermal energy to the contaminated air 102W, and/or to the air 2104 through thermal convection and radiation. The contaminated air flows through clearances and channels formed by materials thermally coupled to the electric heater 2121, which, in turn, transfers thermal energy through thermal conductivity to the materials forming the second heat exchanger 2112. As the contaminated warm air 102W (or 104 for the air sterilizer 2000 configured to operate with UVC lights) flows through the second heat exchanger 2112, thermal energy (heat-temperature) transfers from electric heaters 2121 to contaminated air 102W to increase its temperature and transforms the air into hot or “superheated” sterilized air 103H. The second heat exchanger 2112 can reach controlled and desired temperatures over 100° C., for example, over 400° C. or higher (e.g., 1000° C.) depending on the type of infectious agents to be neutralized (sterilization process), the time the infectious agents contaminated air flows through the internal components forming air sterilizer 2000 (air mass-flow-rate and residence time), and the amount of thermal energy transferred as a given mass-flow of contaminated air 102 transits within air sterilizer 2000. Thermal insulators 2125 surround the heat exchanger and reduce loss of thermal energy to the housing walls as well as protect users from exposure to high-temperature external surfaces of the air sterilizer 2000. In some embodiments, the electric heaters 2121 may be represented by channels where heated fluid from a power source 2112A (thermal or electric power source) is circulated without mixing with the air 102W and/or contaminated air 2104. The power source 2112A may be represented, for example, by waste exhaust gases of a combustion engine, or other equipment that heats up as a result of its operation and rejects thermal energy to the environment. When the invention is configured to operate on vehicles (mobile applications), the equipment that heats up as a result of operations can be represented by internal combustion engine exhaust gases, engine cooling fluids, HVAC working fluids, brake system, electrical motors and generators. Therefore, the power source 2112A represents any waste or directed energy source dedicated to heat up a desired volume of air circulating within scalable air sterilizer 2000.

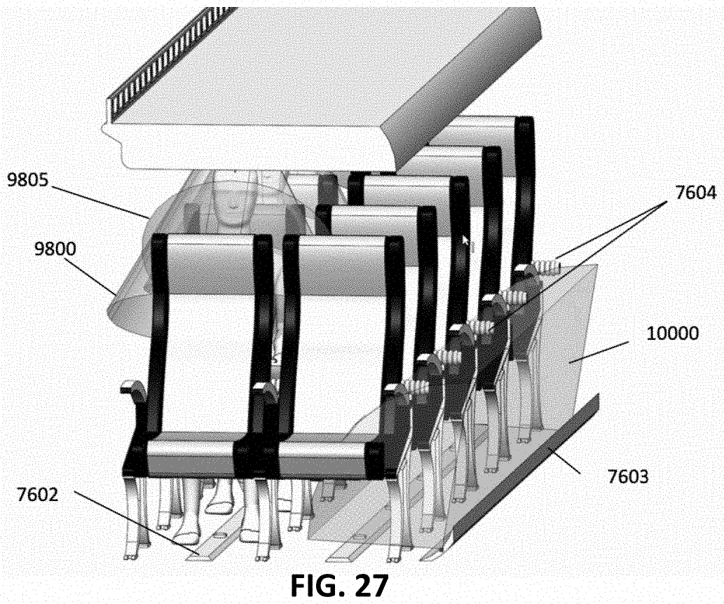

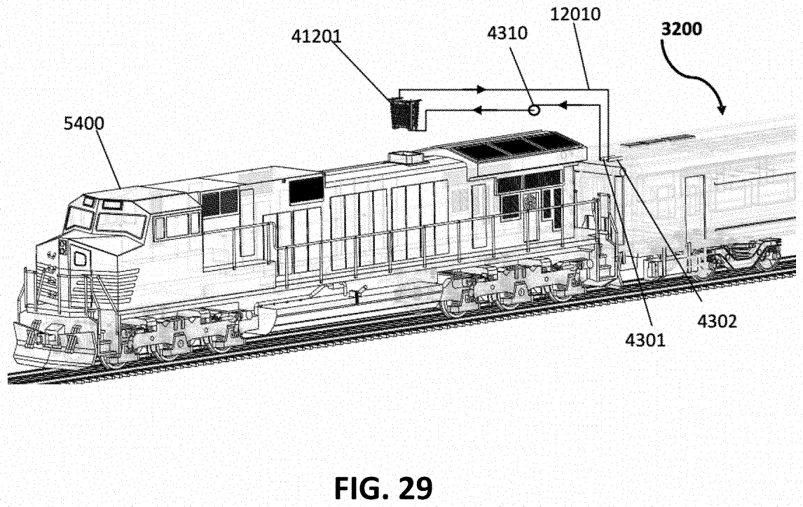

Sterilized hot air 103H continues to flow through the primary side of the first heat exchanger 2110 through the primary channels 2114, for cooling of air 103H (without mixing with contaminated air 102) by heat transfer with colder contaminated air 102 flowing through the secondary side of the first heat exchanger 2110 through the secondary channels 2113. As a result, cooled sterilized air 103 at thermodynamic “state E” (FIG. 2B) exits from the first heat exchanger 2110 and is subjected to further cooling via heat transfer with environmental air (contaminated air 102) via a third heat exchanger 2124. The third heat exchanger 2124 can be formed by fins 2122 thermally coupled to sterilized air 103 on one end and to cooler contaminated air 102 on the opposite end so as to execute cooling functions by natural convection between the contaminated air 102 outside of the air sterilizer 2000 and the sterilized air 103 flowing internally to the air sterilizer 2000. Alternatively, the third heat exchanger 2124 can execute cooling of sterilized air 103 through a combination of natural and forced convection heat transfer with contaminated air 102 without mixing with contaminated air 102 (external to the air sterilizer 2000), via the cooling fan 2123 so as to reset the temperature of the sterilized air 103 at the outlet 2106 to thermodynamic state F as close as feasible to initial thermodynamic state A (FIG. 2B) or to a desired temperature, thus supplying sterilized air 103 at adequate ambient temperature for inhalation. Sterilized air 103 exits the air sterilizer 2000 through one or multiple outlets 2109, which may include a plurality of nozzles configured to shape jets of the sterilized air 103. Sterilized air 103 may be distributed by air-conditioning duct systems which may be retrofitted with specialized sterilized air delivery systems that generate “air curtains” 2800 to shield individuals and groups of individuals from contaminated air as shown, for example, in FIGS. 27 and 29. For configurations of the air sterilizer 2000 equipped with UVC lights 2200, ozone recombiners 2202 may be utilized to intercept the sterilized air 103 that might still contain ozone that did not decay due to high temperature exposure when warm air 102W is circulated through heating elements 2121.

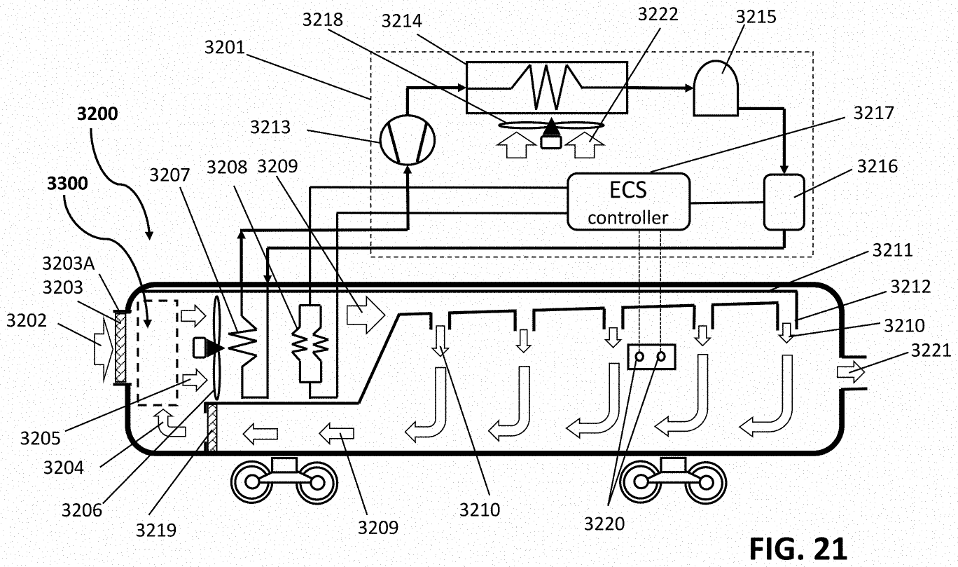

In FIG. 21, a passenger railcar 3200 is shown with a simplified schematic diagram of an Environmental Control System and HVAC system 3201 normally operating in these vehicles.

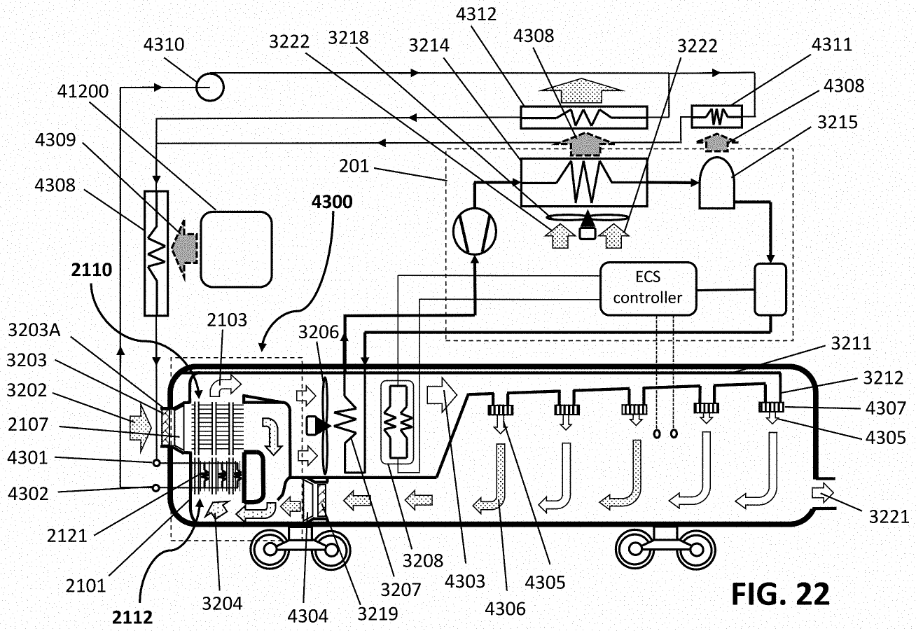

Accordingly, fresh air 3202 flows through a fresh air filter 3203 and mixes with recirculated air 3204 as a result of an operating blower 3206. The blower 3206 circulates a mixture of recirculated and fresh conditioned air 3209 through an ECS/HVAC duct system 3211, which includes an evaporator 3207 and heating elements 3208 configured to cool or heat the air respectively based on the settings of an ECS control system 3217. As conditioned air 3209 flows through the duct system 3211, it is distributed through the railcar cabin or interiors through duct openings 3212 that dispenses conditioned air 3210 based on settings of a thermostat 3220. A certain volume of the conditioned air 3210 is then bled through bleed air 3221, and an equivalent volume of air inlets the passenger railcar 3200 through the fresh air filter 3203. Recirculated air 3204 is filtered by a filter 3219 and mixes with the fresh air 3202 to form pre-conditioned air 3205. Portion of the ECS/HVAC equipment may be included in the system 3201, The compressor 3213 suctions a suitable working fluid (e.g., organic fluid), compresses it into the condenser at a high pressure. The working fluid flows through a receiver 3215 and an expansion valve 3216 prior to entering the evaporator 3207 as it is traditionally done in refrigeration cycles. Depending on the settings of the thermostat 3220 and an environmental temperature, the conditioned air 3209 is either cooled down or heated to a comfortable temperature. A condenser 3214 is cooled down by thermal transfer to environmental air 3222 through forced air convection via a fan 3218. This represents waste thermal energy that can be recovered for supporting the superheating functions of the air sterilizer implemented in the railcar 3200. The air sterilizer may be scaled and retrofitted within the passenger railcar at any locations, for example, within the ECS/HVAC equipment compartments and ducts as indicatively represented by a retrofitted equipment compartment including the majority of an air sterilizer 4300 shown in FIG. 22.

With reference to FIG. 22, the fresh air 3202 enters the railcar at an inlet 3203A, and enters the air sterilizer 4300, flowing through the first recuperator heat exchanger 2110 as described in FIG. 20. Fresh air 3202 enters the air sterilizer 4300 through the compressor 2107 supporting the suctioning effect generated by the blower 3206. As described above in connection with FIG. 20, the air sterilizer 4300 superheats a recirculated air 3204 (which may contain infectious agents) and the fresh air 3202 to generate the sterilized air 2103. The sterilized air 2103 is then placed into circulation inside the railcar duct system 3211, flowing through the evaporator 3207 and the heating elements 3208 to become a conditioned sterilized air 4303. This sterilized and conditioned air 4303 is then distributed by the duct openings 3212 and sterilized air through nozzles 4307. The nozzles 4307 are configured to generate films of sterilized and conditioned air 4305 (hereafter referred to as air curtains 4305). The air curtains 4305 provide zoned air shields intercepting airborne infectious agents and blocking these agents from circulating throughout the passenger railcar cabin internals. In some embodiments, the air sterilizer 4300 utilizes waste thermal energy from railcar HVAC/ECS equipment as shown in FIG. 22. Accordingly, the heating elements 2121 may be replaced or configured to work in tandem with a working fluid circulated through a pump 4310 in a closed loop. The working fluid may be any suitable fluid that remains stable at relatively high temperatures (e.g., organic, water-steam, oil). In this configuration, the working fluid is circulated from a heat source (e.g. a waste heat source) as represented by the condenser 3214 and the receiver 3215 which may be thermally coupled to auxiliary heat exchangers 4311, 4312, and 4308. The heat exchangers may be coupled to any source of waste thermal energy which, in the context of a railcar, may be represented by equipment or waste heat energy sources 41200 that reject thermal energy to the environment (dynamic brakes, traction motor, HVAC/ECS, battery power conditioning systems, or combustion gases from the internal combustion engine equipping diesel-electric locomotives). The heat recuperated through the auxiliary heat exchangers 4311, 4312, and 4308 is then transferred by the working fluid to the second heat exchanger 2112 of the air sterilizer 4300, in which the heating elements 2121 may be replaced by channels in which the working fluid flows, or they can be coupled to further increase the temperature of recirculated air 3204 and fresh air 3202 should the thermal energy recovered be insufficient to assure sufficiently high temperature to neutralize infectious agents possibly contained in the air.

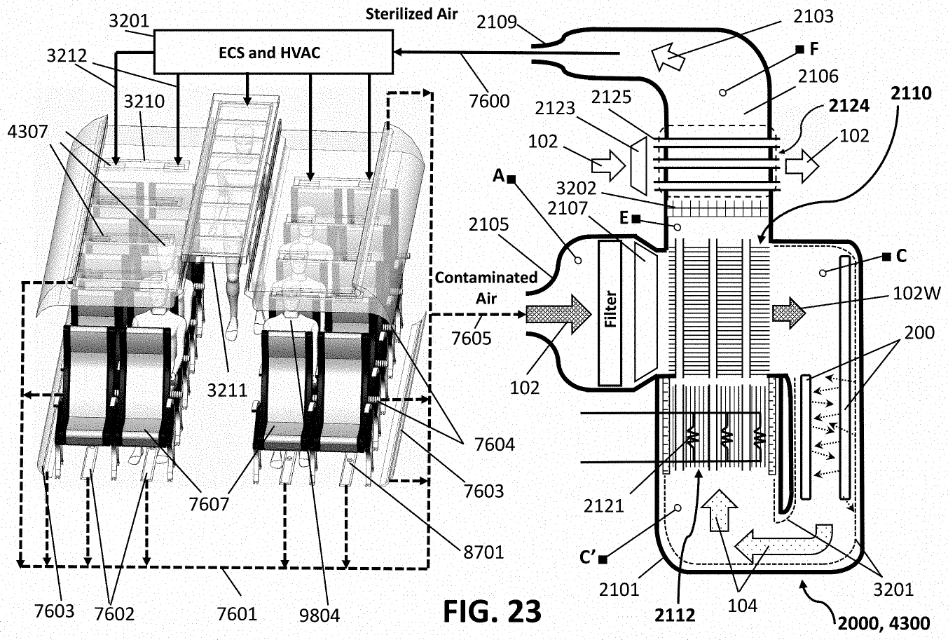

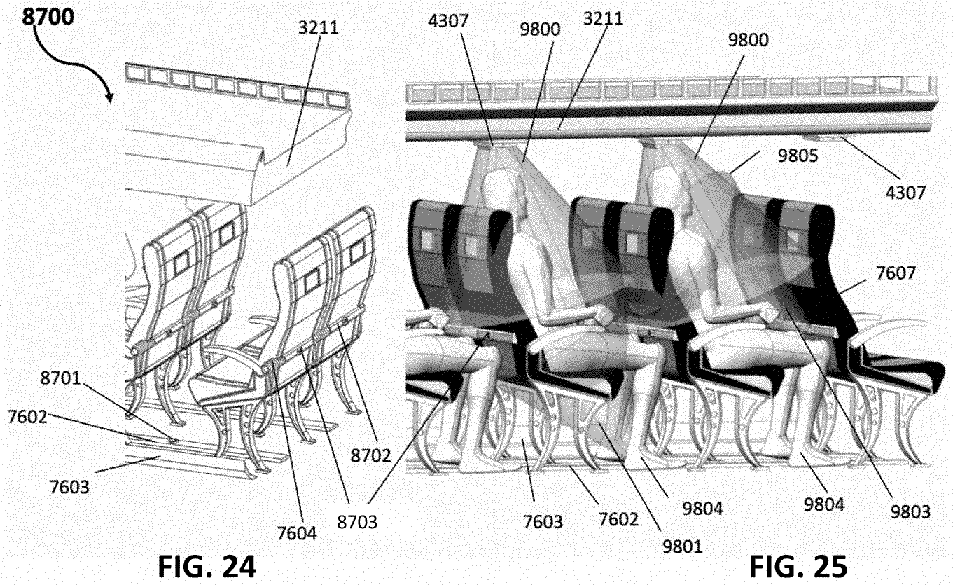

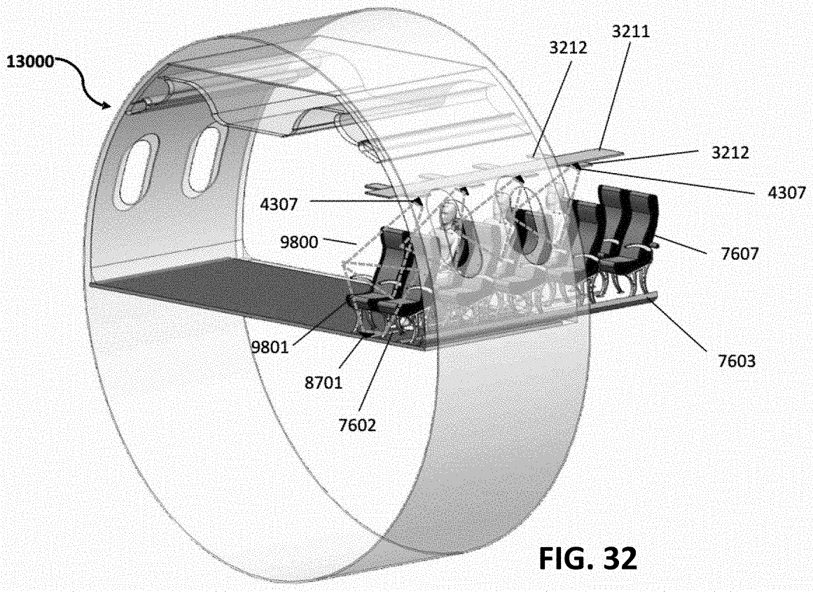

FIG. 23 illustrates an application of the air sterilizer 2000 described in FIG. 20 or the air sterilizer 4300 as described in FIG. 22 as an apparatus utilized to sterilize the air circulating within a closed environment represented, for example, by the cabin of a passenger railcar, an aircraft or a bus as shown in FIGS. 32, 33, and 34, respectively. In this configuration, the ECS/HVAC ducting system 3201 distributes sterilized air 2103 through a duct 7600 and duct conduits 3212 to deliver conditioned air (or curtains of sterilized air) 3210 via nozzles 4307. As sterilized air 3210 circulates within the closed environment represented by the interiors of a railcar (or aircraft fuselage, bus cabin and any other transport platform), it is recaptured for high-temperature re-processing through the air sterilizer 2000 or 4300 as contaminated air 7605. The contaminated air 7605 is suctioned by various suctioning ports positioned within suctioning ducts 7601. Suctioning ports may be configured as openings 8701, 7604, 8703 shown, for example, in FIGS. 24- 25. The suctioning ports may be positioned within suctioning ducts or bottom strips 7602, lateral bottom strips 7603, seat strips 8702 (FIG. 24). The suctioning ports 8701, 8700 and those positioned within the lateral strip 7603 is to generate a depression zone closely located to each individual seat so as to capture potentially contaminated air exhaled by individual passengers 9804 which may contain airborne infectious agents. The nozzles 4307 are configured to distribute sterilized air forming a “protective air bubble” or air curtain 9800 as shown in FIG. 25.

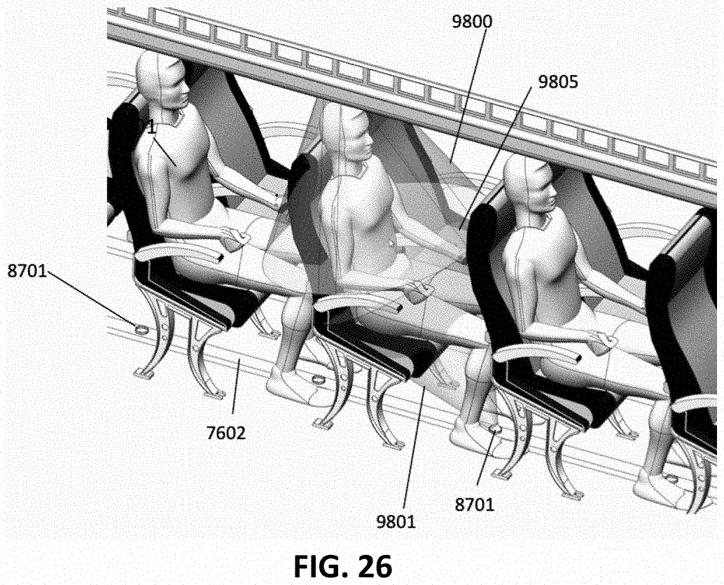

FIGS. 24 and 25 illustrate locations for contaminated air suctioning ports 8701, 8700, 7604 and those disposed at the lateral bottom strips 7603, and sterilized air curtains 9800. The depression areas generated in the lower and frontal portions of each passenger seat are configured to intercept and capture airborne infectious agents potentially emitted by individual passenger 9804, and circulate them along with the bulk of contaminated air 7605 (see FIG. 23) back at the inlet 2105 of the air sterilizer 2000 or 4300 for re-sterilization. The combined effects of the sterilized air curtain 9800, with the depression areas 9801, 9805 (FIGS. 25 and 26) and 10000 (FIG. 27), generated by suctioning ports distributed along contaminated air collection strips (e.g., 7602, 7603, 8702) effectively create an “air bubble” which provides sterilized air to each passenger and decontaminates the air and airborne infectious agents that may be emitted by each passenger.

FIG. 26 illustrates in greater detail the air bubble formed by combining the air curtain 9800 with the suctioned air depression areas 9801 (only shown operational for one seat for simplicity).

FIG. 27 illustrates in greater detail the air bubble formed by combining the air curtain 9800 with the suctioned air depression areas 9805 (positioned in the back seat and in front of each passenger) and the depression areas caused by suctioning ports positioned on suctioning strip 7603 hydraulically coupled to the return line suction ducts 7601 shown in FIG. 23.

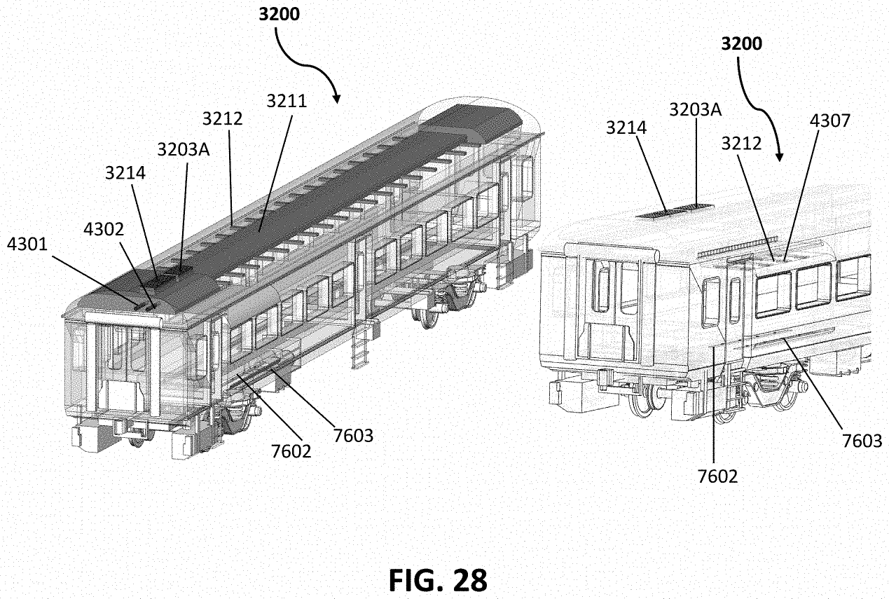

FIG. 28 illustrates the positioning of high-temperature outlet ports 4301 and 4302 configured to couple to a waste heat source (41200 and the waste heat exchanger 41201 shown in FIGS. 22 and 29), also represented by the condenser heat rejection vent of the condenser 3214 (FIG. 22), and the general locations of the suctioning strips 7602 and 7603 (FIG. 28). In this figure, the general locations of the sterilized air nozzles 4307 and their ducts included in the ECS/HVAC duct system 3211 and the duct openings 3212 are also shown.

FIG. 29 illustrates an application of the air sterilizer 2000 or 4300, in which the waste heat source is represented by the exhaust gases of a diesel-electric locomotive 5400. In this configuration, a high temperature working fluid is thermally coupled to a heat exchanger 41201 on one end and to the heating elements 2121 of the air sterilizer 2000 or 4300 on the other end, by high temperature flexible lines 12010 which bridge and connect locomotive 5400 to railcars 3200 (each thermal-hydraulically connected to similar high-temperature flexible lines via outlet ports 4301 and 4302).

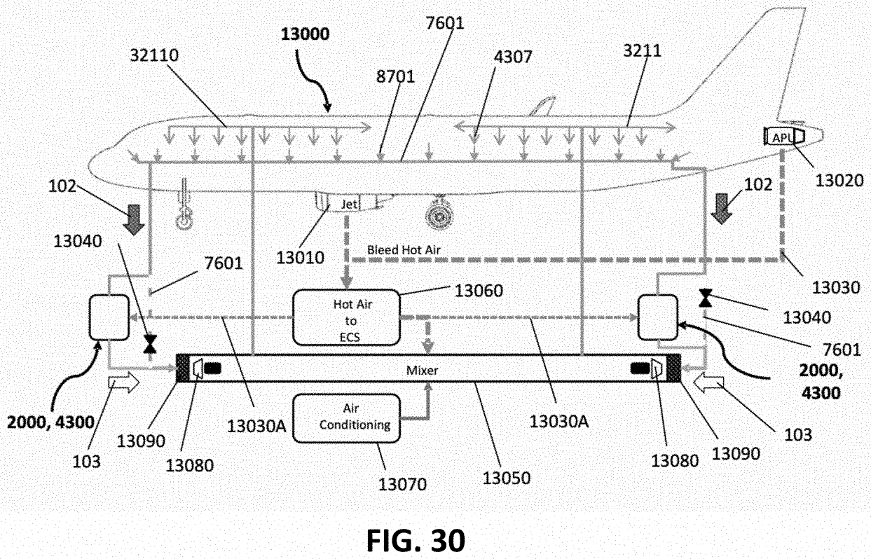

FIG. 30 represents another application of the air sterilizer 2000 or 4300 configured to sterilize the air circulating within the fuselage of a passenger aircraft 13000. In this configuration, the air sterilizer 2000 or 4300 is thermal hydraulically coupled to the aircraft ECS duct system. As shown in FIG. 30, the contaminated air 102 from the suction ports 7601 along the fuselage air collection line is processed within the air sterilizer 2000 or 4300, and the sterilized air 103 is provided at the inlet of an ECS mixer 13050. The waste heat source utilized to elevate the temperature of the heating elements 2121 within the second heat exchanger 2112 is represented by thermal coupling with the high temperature exhaust gases generated by the jet engine (in flight) and/or by an Auxiliary Power Unit (APU) 13020, for example, during boarding operations, as air from the airport terminal also enters the aircraft during the boarding operations. The principles of operation of the air sterilizer 2000 or 4300 are similar to those described above in connection with FIGS. 20-27 and the methods of dispensing sterilized air through the cabin of the aircraft 13000 are the same as those described in connection with FIGS. 23-27.

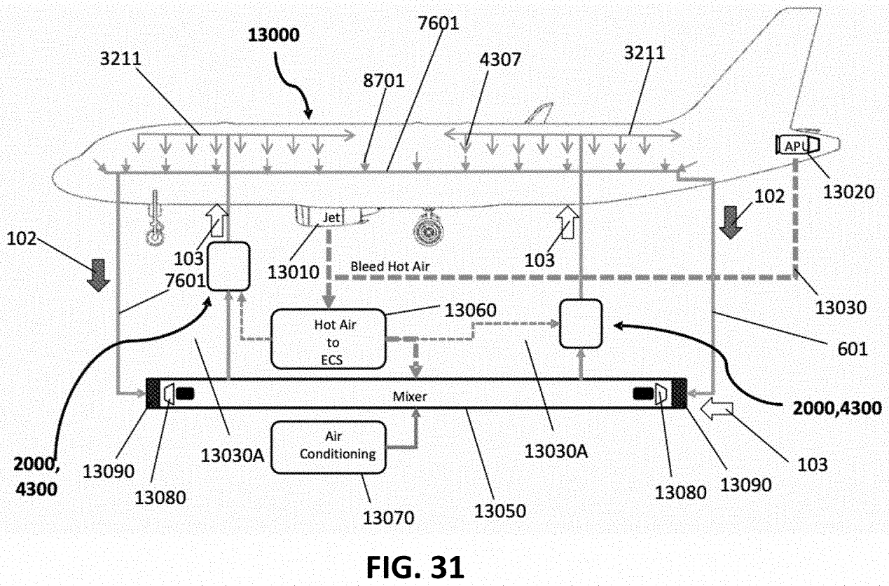

FIG. 31 represents another application of the air sterilizer 2000 or 4300 configured to sterilize the air circulating within the fuselage of the passenger aircraft 13000. In this configuration, the air sterilizer 2000 or 4300 is thermal-hydraulically coupled to the aircraft ECS duct system on the duct line providing ECS filtered air to the fuselage of the aircraft 13000. As shown in FIG. 31, the contaminated air 102 from the suction ducts 7601 of the fuselage air return/collection line is processed first by the mixer of the traditional ECS and then by the air sterilizer 2000 or 4300, so that sterilized air 103 is provided at the air-curtain forming nozzles 4307 (FIG. 25). The waste heat source utilized to elevate the temperature of the heating elements 2121 within the second heat exchanger 2112 (FIG. 22) is represented by thermal coupling with the high temperature exhaust gases generated by the jet engine (in flight) and/or by the Auxiliary Power Unit (APU) 13020, for example, during boarding operations. The heating elements 2121 of the second heat exchanger 2112 may also be supported by electrical heating. The principles of operation of the air sterilizer 2000 or 4300 in this embodiment are similar to those described above in connection with FIGS. 20-27 and the methods of dispensing sterilized air through the cabin of aircraft 13000 are the same as those described above in connection with FIGS. 25-29.

FIG. 32 illustrates a perspective see-through portion of the fuselage of aircraft 13000. Accordingly sterilized air is distributed through the nozzles 4307, which are configured to generate the sterilized curtain 9800 substantially surrounding each seat/passenger and deflecting surrounding contaminated air for this contaminated air to be suctioned via the aircraft ECS return lines. Similarly as shown in FIGS. 23-27, the suctioning ports (e.g., 8701) are positioned to capture potentially contaminated and airborne infectious agents, generated by one or more passengers, by suctioning air through the depression zones 9801 to recirculate the captured air back into the air sterilizer 2000 or 4300 integrated with the ECS and HVAC systems of the aircraft.

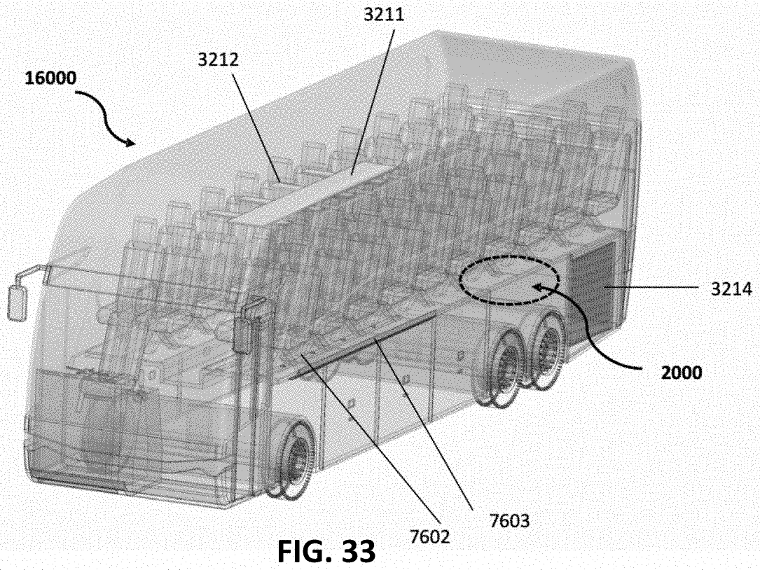

FIG. 33 illustrates another application of the air sterilizer 2000 or 4300 integrated with an automotive, such as a bus 16000. The functioning principles of the air sterilizer 2000 or 4300 applied to the transport platform represented by the bus 16000 are similar to those described above in connection with FIGS. 20-24, and the waste thermal energy source 2112A is represented by coupling the heating elements 2121 of the second heat exchanger 2112 to the exhaust gases generated by the internal combustion engine of the bus 16000.

The air sterilizer can be implemented to any vehicle, including a military vehicle, special purpose vehicle (e.g., vehicles for research purposes), a public or commercial transit vehicle (such as an aircraft, a train, a bus, a ship, etc.) to neutralize airborne infectious agents by sterilizing the air recirculated through the cabin in real time. That is, the sterilization of the air can be performed while the vehicle is in operation with customers or crews present in the vehicle. The system includes high-effectiveness heat exchangers to execute real-time air sterilization. The system exposes the contaminated air to high temperatures to irreversibly damage and neutralize infectious agents (including natural and man-made viruses), and quasi-simultaneously cools the sterilized air down to ensure unimpaired operation of the ECS and HVAC equipment. The disclosed method includes increasing, quasi-instantaneously (or in real time), a temperature of potentially contaminated air and cooling it down as it exits the recuperator heat exchanger of the air sterilizer. The present disclosure provides the ability of real-time disinfecting the air in a vehicle, with no side effects as compared to other disinfecting technologies, and no maintenance and safety protocol requirements for handling specialized filters.

Protection of the driver area of a public transit vehicle via high-temperature air sterilization and slight pressurization of the area surrounding the driver by means of localized components of the disclosed air sterilizer can be configured to deflect infectious airborne agents directed at the driver (e.g., via unshielded sneezing of passengers), thus creating a sterilized air bubble surrounding and protecting the driver at all times.

The operations of the disclosed air sterilizer do not rely on filters, and therefore, do not require frequent filter changes that could expose operators to infectious agents, and do not cause potential harm from exposure to harsh chemicals and fumes, do not induce damage to skin and eyes from UV-C exposure, do not produce toxic ozone or harmful chemicals, do not alter air parameters such as humidity and oxygen content and do not affect on-board systems. The disclosed air sterilizer provides a viable, simpler, maintenance free and safer sanitation approach for the public or commercial transportation.

The present disclosure provides a method to neutralize airborne infectious agents using the vehicle waste heat sources, as there are no real-time air sterilization methodologies that can be used during vehicle operations (in the presence of passengers and operators). Although specialized filters could be considered real-time mechanisms that trap viral loads, these special filters also concentrate viral loads. Some filters may be manufactured with virus neutralizing or containment components which tend to increase operating costs due to increased replacement frequency, which also can increase operating hazards during replacement.

The disclosed air sterilizer utilizes waste heat source and/or electricity. For embodiments in which electrical heat sources are used, the “recuperator” heat exchanger lowers the electric consumption as the hot sterilized air is recirculated back into the air sterilizer to heat up the incoming colder infectious-agents contaminated air, thereby substantially reducing the electrical load on the vehicle's power supplies.

Overall, the disclosed air sterilizer can be configured to be retrofittable with the HVAC components of the transit vehicles for mobile applications. The disclosed air sterilizer can be scalable to match different vehicle models, air flow rates, dimensional and heat source requirements. The disclosed air sterilizer can retrofit components interfaced with flanges, fittings and adapters to supports rapid replication by transit agencies. The disclosed air sterilizer can be replicated for applications on different transportation modes by retrofitting the HVAC, Environmental Control Systems (ECSes) and Air Handling components included in a public transit vehicle, such as a passenger railcar, a cruise ship. Localized contaminated air sterilizer can be used for cabins and centralized contaminated air sterilizer can be used for ship mall, restaurants, engine and crew environments, and aircraft cabins, where the recuperator heat exchanger of the system is thermally coupled to the aircraft Auxiliary Power Unit (APU) exhaust gases during boarding/airport operations, and to the aircraft jet engines during flight.

The present disclosure provides simple and cost-effective air disinfection methodologies to increase passengers and operator safety for all transport platforms, without increasing operating costs.

One aspect of the disclosure provides a retrofittable contaminated air sterilization system, device, or apparatus (or referred to as air sterilizer) configured to circulate air potentially containing infectious agents (including SARS-CoV-2) through the primary side of a heat exchanger coupled to a heat source to increase the temperature of the contaminated air. By increasing the temperature of this air, the air sterilizer irreversibly denatures the proteins forming infectious agents. The superheated air is then circulated back to the secondary side of the heat exchanger to transfer its energy to the colder air circulating through the primary side of the heat exchanger. With this configuration the energy required to heat up the potentially infected air is substantially reduced as the heat exchanger is effectively functioning as a “recuperator” heat exchanger.

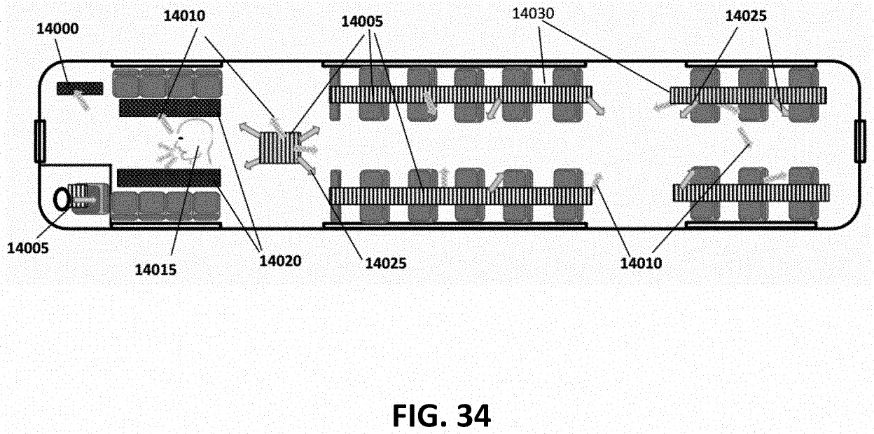

FIG. 34 shows a transit bus simplified cross-sectional top-view indicating the general air ventilation configuration (part of the HVAC system) with comfort air vents 14005 and return air vents 14020 recirculating the air within the cabin by removing and re-supplying it to passengers and driver during normal operations.

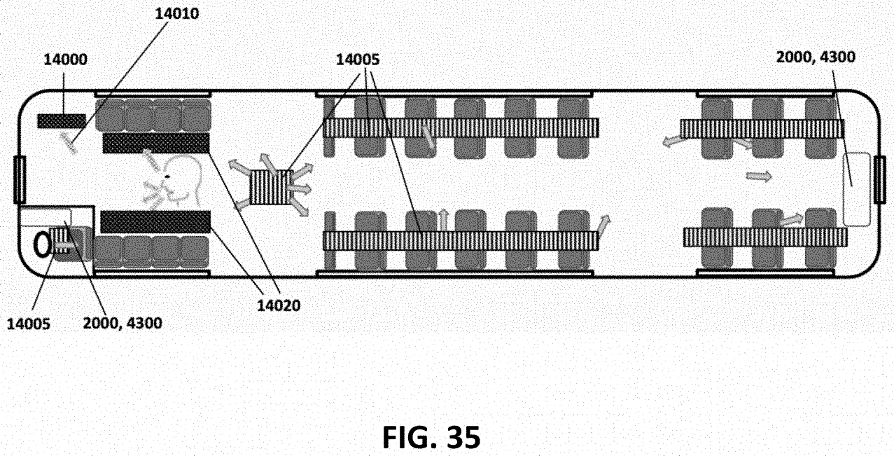

FIG. 35 illustrates mechanisms of contaminated aerosols generated, for example, near the driver area by an infected carrier 14015. As a result of, for example, a passenger sneeze, and assuming this particular passenger is infected with SARS-CoV-2, the air in the cabin becomes contaminated with airborne SARS-CoV-2 particles 14010. On average a single sneeze from an infected person represents approximately 2,000,000 SARS-CoV-2 nano-particles aerosolized and airborne. These airborne particles mix with the air inside the cabin which then travels through inlets and the return air vents for the HVAC system (e.g., 14030) to partially filter and increase or decrease its temperature prior to distributing it back through the comfort air outlets 14005 positioned throughout the vehicle. As a result, SARS-CoV-2 particles spread. In the example shown in FIG. 34, air with infectious agents spreads from the seats in the front of the vehicle to those in the back by means of the HVAC operation. In FIG. 34, 14025 represents the HVAC processed air, and 14035 represents particles distributed by the HVAC system.

FIG. 35 illustrates that infectious agents are no longer distributed by the HVAC system as they are intercepted while circulating through the return air vents 14020 and sterilized by exposure to a high temperature that damages and inactivates the proteins forming infectious agents. The high-temperature air is then cooled down through a dedicated heat exchanger retrofitted with the HVAC components (included in the air sterilizer 2000 or 4300) and reset the sterilized air temperature to a comfortable temperature prior to being redistributed through the comfort air vents 14005.

The solution to air disinfection to mitigate passengers and operators' exposure and to reassure public confidence in transit involves a technology or method that actively neutralizes airborne infectious agents generated by any of the vehicle occupants as the air circulates through the vehicle HVAC system. The real-time airborne infectious agents neutralizer (i.e., air sterilizer) disclosed herein is a technology that processes and neutralizes airborne infectious agents by sterilizing the air in real time. The air sterilizer needs only low maintenance and employs very few components, mainly a high effectiveness stainless steel heat exchanger coupled to a heat source, an electronic controller and an air-fan to boost air circulation within the heat exchanger as shown in FIGS. 1 and 20. These components can be scaled to process a desired amount of air and geometrically adapted to non-invasively, or minimally invasively, be retrofitted with the components normally equipping the vehicle HVAC system. These components can be similar to those included in the air sterilizer 2000 or 4300 described above in connection with FIGS. 20-35, the description of which are not repeated. The heat source can be electrical (e.g., electrical heaters) or waste thermal energy from vehicle operations (e.g., HVAC condenser, regenerative braking, traction motors or combustion engines exhaust gases and cooling systems).

More specifically, the system executes quasi-instantaneous thermodynamic superheating and cooling of the air as it flows through the “recuperator” heat exchanger included in the air sterilizer 2000 or 4300. Air potentially carrying infected agents enters the primary side of the heat exchanger where it is heated to a temperature of 200° C. (392° F.) or higher. As the air is heated, the infectious agents contained in it are neutralized mainly by protein denaturation. Very hot disinfected air is then circulated into the secondary side of the heat exchanger so as to recover part of the energy employed to heat up the incoming infected air. This configuration enables a partial recuperation of the energy involved in heating up the air while cooling down the disinfected air prior to discharging it back to the HVAC system for processing. The HVAC system then executes temperature adjustments prior to distributing the air through the vehicle comfort air vents 14005. These features significantly lower the energy consumption of the air sterilizer 2000 or 4300 when its heat source is represented by electrical heaters. As components of the air sterilizer 2000 or 4300 can be scaled, the air sterilizer 2000 or 4300 can be tailored and retrofitted with the multiple HVAC systems providing localized comfort air (e.g., driver area) or centralized comfort air to passengers.

The air sterilizer 2000 or 4300 provides continuous/full-time sterilization of infectious agents during vehicle operations. Currently adopted methodologies dedicated to neutralizing infectious agents are represented by specialized filters, UVC lamps and chemical disinfectants on surfaces applied only when passengers are not in the vehicle. Filters require frequent replacement and represent maintenance hazards and cost. UVC light cannot be operated when passengers and operators occupy the vehicle as ultraviolet-C radiation is harmful to tissues and eyes and also generates toxic ozone which may require about two days to decay back to oxygen. Chemical disinfectants are effective on surfaces. However, a clean surface is compromised as soon as an infected individual comes into contact with such surface. Additionally, chemical residues can be toxic and tend to damage equipment. Finally, these currently adopted methodologies represent increased operating costs.

The air sterilizer 2000 or 4300 simply burns and cools down the air as the air is recirculated through the vehicle's centralized and localized HVAC systems. No chemical residues and no ozone are generated. In addition, no specialized filter replacement, and no maintenance is required.

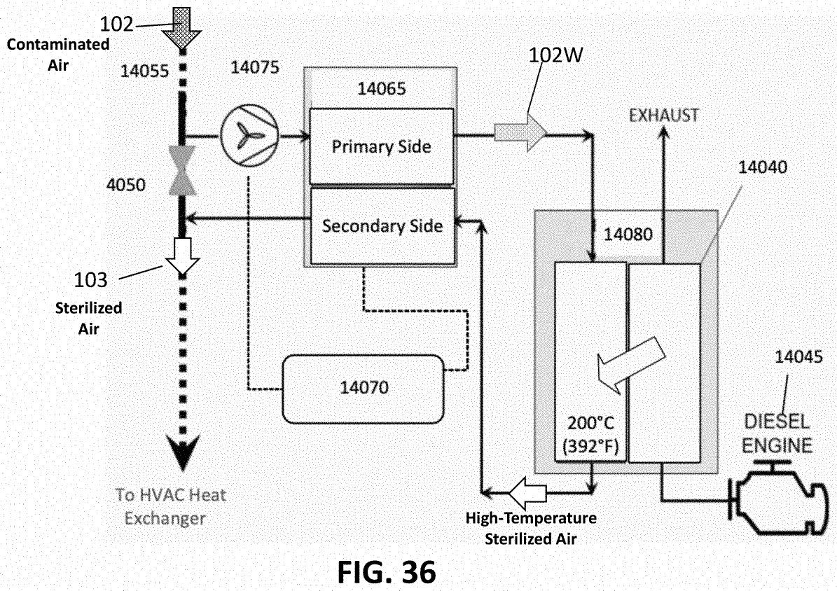

FIG. 36 illustrates the main components of the air sterilizer 2000 or 4300. The air from the HVAC air return duct 14055 is intercepted, circulated by a fan 14075 through the primary side of a recuperator heat exchanger 14065 and a heat source heat exchanger 14080 to increase its temperature, and back to the secondary side of the recuperator heat exchanger 14065 to lower the now sterilized air temperature prior to returning it to the vehicle HVAC system. For configurations in which a heat source 14045 utilizes waste heat energy from the operation of the vehicle's engine, the electric consumption is represented by the fan 14075 and an electronic controller 14070. The fan 14075 is utilized to compensate for a potential back pressure induced by the recuperator heat exchanger 14065 and the heat source heat exchanger 14080. It is anticipated that as the backpressure represented by these components is comparable to that caused by specialized filters to reduce viral loads, the air sterilizer 2000 or 4300 may operate even without a fan.

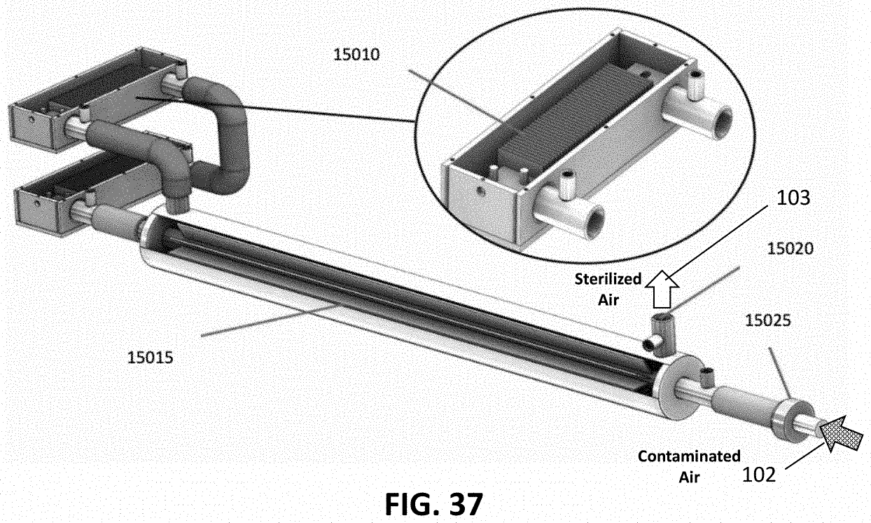

As shown in FIG. 37, air potentially containing infectious agents is quasi-instantaneously superheated by thermally coupling it to a relatively high temperature heat source, effectively sterilizing it. Heated surfaces of the air sterilizer 2000 or 4300 are thermally coupled to electrical heaters and/or waste thermal energy from the combustion engine (e.g., bus diesel engine). As shown in FIGS. 34-35, infectious agents potentially generated by a carrier tend to recirculate throughout the confined environments inside the vehicle, thus favoring spreading from one host to another. FIG. 36 illustrates a simplified diagram of the working principles of the air sterilizer 2000 or 43000 based on a few main components: the recuperator heat exchanger 14065 for air mixed with infectious agents (contaminated air) to be exposed to high temperatures as it flows through the primary side of the recuperator heat exchanger 14065 and the heat source 14045 supplying thermal energy to the secondary side of the recuperator heat exchanger. The recuperator heat exchanger 14065 is configured such that contaminated air intercepted from the bus return air vents flows in close contact with the heat exchanger's surfaces. Thus, the temperature of the air increases as a result of thermal transfer from the heat exchanger surfaces to the air flowing through the recuperator heat exchanger. The heat source 14045 (electrical or waste energy from the operation of the vehicle) provides thermal energy to the heat transfer surfaces. The process involves contaminated air at an initial temperature flowing though the inlet of the heat exchanger of the recuperator 2000 or 4300. As air flows into the heat exchanger, its temperature increases to a desired value (e.g., 200° C.). Potentially infected air is initially heated as it flows through the primary side of the recuperator heat exchanger 14065 as a result of heat transfer from the sterilized hot air returning from the heat source heat exchanger 14080 and flowing through the secondary side of the recuperator heat exchanger 14065. As air exits the primary side of the recuperator heat exchanger 14065, it enters the heat source heat exchanger 14080, where its temperature is further increased to a desired value between a room temperature to a temperature greater than 100° C. (200° C.-400° C.). This superheated air desiccates and denatures SARS-CoV-2 virus and other infectious agents. This superheated and sterilized air is then circulated through the secondary side of the recuperator heat exchanger 14065, where it is cooled by the infected air entering the primary side of the heat exchanger 14065 without mixing. The electronic controller 14070 may be configured to regulate the flow rate by changing the compressor parameters, or the heat transfer between the heat source and the infected air volume to be treated. The electronic controller 14070 regulates the mass-flow-rate of air flowing through the inlet of the air sterilizer 2000 or 4300, the temperature of the heat source, the heat transfer from the heat source.

To maximize adaptability to different HVAC systems equipping different bus models, the air sterilizer 2000 or 4300 features multiple heat exchangers configurations designed to reduce retrofitting invasiveness and cost. Contaminated aerosols follow air drafts and tend to collect altogether with the air circulating within the return ducts. The air sterilizer 2000 or 4300 intercepts the return air to the HVAC systems and super heats it to a temperature that neutralizes infectious agents including SARS CoV-2, MERS, measles morbillivirus, chickenpox virus, mycobacterium tuberculosis, influenza virus, enterovirus, norovirus, adenovirus, and syncytial virus — effectively neutralizing all infectious agents that spread through airborne transmission.

The temperature provided by the air sterilizer 2000 or 4300 may be set to 200° C. (392° F.) or higher to ensure ample safety margin and decrease time of exposure to seconds or a fraction of a second as potentially contaminated air flows through its heat exchangers. The maximum temperature can further be increased (e.g., to 400° C., 752° F. or higher). Further increasing the maximum temperature does not impact operating cost when the heat source is waste energy from operation of the vehicle (e.g., exhaust gases and engine cooling system). If the air sterilizer 2000 or 4300 uses an electrical heating heat source, its energy consumption and operating cost increases proportionally to temperature increases. The temperature of 200° C. is most likely overly conservative. Resetting this temperature from any environmental value to 400° C. may be achieved by reprogramming in the electronic controller 14070 (as setting a household thermostat).

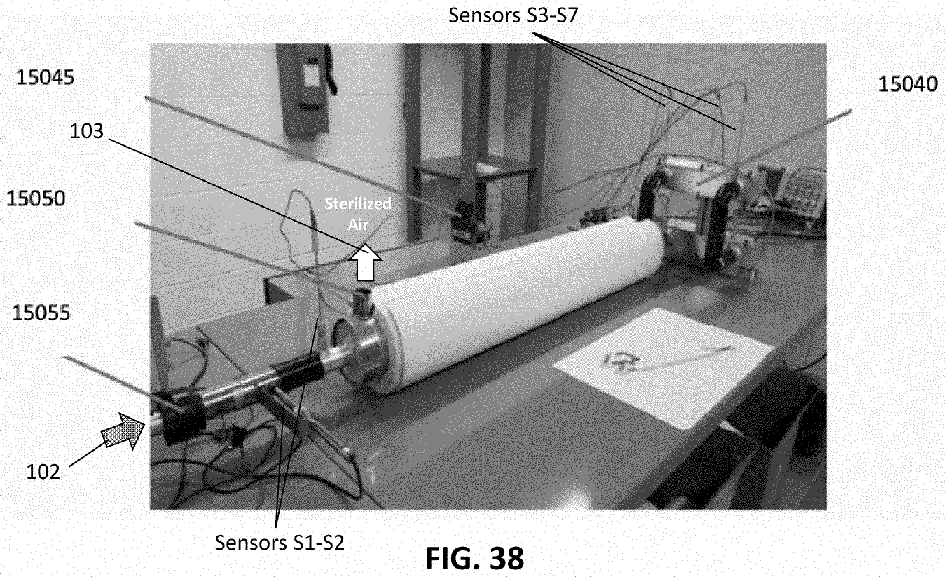

A low-cost prototype of the air sterilizer 2000 or 4300 utilizing off-the-shelf heat exchangers is shown in FIGS. 37-38. A finned tube heat exchanger 15015 (also referred to as a recuperator heat exchanger 15015) was used to benchmark thermodynamic performance parameters to support computer modeling and codes validations. The prototype shown in FIGS. 37-38 utilizes electrical heaters 15010 as a thermal source. The reference number 15020 is an exhaust port, and the reference number 15025 is an inlet circulation fan. For testing, this small-scale prototype sterilized the volume of air of a traditional office room by plugging it in to the office electrical outlets.

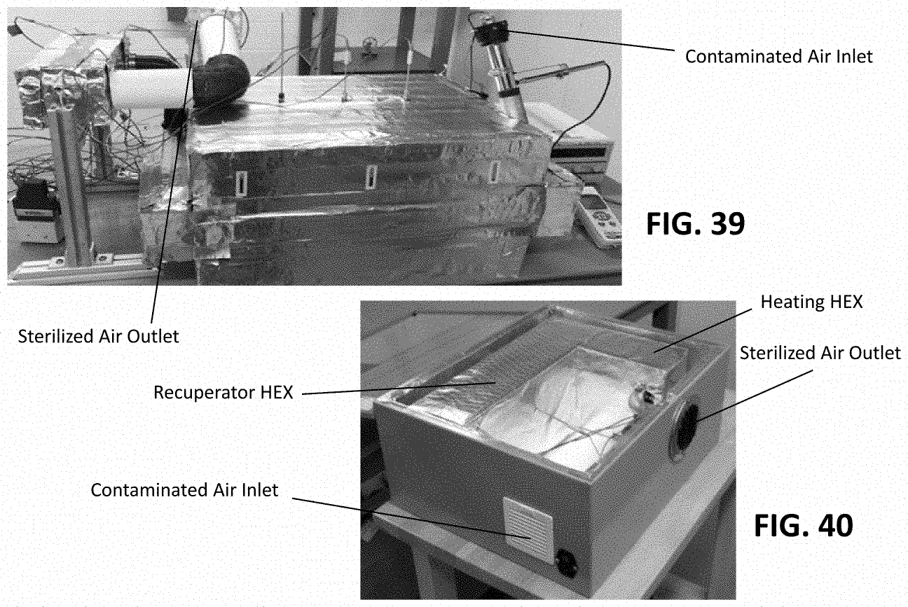

FIGS. 38-39 show prototypes of the air sterilizer 2000, 4300 with different scales and type of heat exchangers to process an increased volume of contaminated air tested to increase economic model accuracy. For all air sterilizer 2000, 4300, the temperature at an outlet of the electrical heater 15010 may be set to a desired value to ensure real-time air-sterilization. FIG. 38 shows the experimental setup, to measure performance of the air sterilizer 2000, 4300. In this Figure, 15040 represents the housing of heaters 15010 shown in FIG.37, 15045 represents controller 300, 15050 represents sterilized air outlet 103, and 15055 represents motor-driven compressor fan 107.

Based on the prototypes developed and tested for the air sterilizer 2000 or 4300, components manufacturing and assembly do not represent costing and manufacturing challenges, can be mass-produced with conventional manufacturing equipment and scaled to comply with dimensional and HVAC equipment requirements. The components can be scaled to process different air flow rates, heat sources and satisfy specific requirements (e.g., residence time, max power rating). For example, assuming an average vehicle internal volume of 960 ft³ (−27 m³) and an electric power consumption capped at 1 kW (electrical heat source configuration), the air sterilizer 2000 or 4300 would take approximately 18 minutes to sterilize this volume of air. By increasing the electric power rating, the time to process this volume of air proportionally decreases. A typical transit bus is equipped with a combustion engine producing 200-450 HP (150-335 kW) with an equivalent power rating represented by waste thermal energy via exhaust gases and water cooling. Engines equipped with pollution reduction technologies require high temperature exhaust gases. Assuming only 30 kW of thermal energy is extracted from the exhaust gases (over 100 kW will be available from any transit engine model), the air sterilizer 2000 or 4300 may be configured to process the whole cabin volume in less than 1 minute. To minimize impact on transit bus Original Equipment Manufacturer (OEM) components, components of the air sterilizer 2000 or 4300 may be retrofitted with dimensions to match fitting, flanges, balance of plant and equipment housing requirements.

The air sterilizer 2000 or 4300 can be operated at all times during public transit vehicle operations and its operational cost varies whether the unit is powered by electricity or waste heat sources. The air sterilizer 2000 or 4300 does not require maintenance and its components will be designed to operate for at least 10 years without requiring refurbishing or replacement. Operating cost nears zero when waste heat sources are utilized. As current HVAC systems are operating with specialized filters causing increased back pressure, the reduction in filter usage enables the OEM HVAC fan(s) to operate the air sterilizer 2000 or 4300 without need for the additional fan shown in FIG. 36, thus reducing operating cost nearly to zero. The electronic controller power consumption of the air sterilizer 2000 or 4300 can be less than 50W-equivalent to a halogen headlight bulb power consumption.

FIG. 40 shows the general fuel potential repartition as transit buses equipped with internal combustion engines offer a substantial waste energy source. Similarly, the air sterilizer can be applied to marine vessels and aircrafts. Assuming, for simplicity, an average engine efficiency of approximately 33%, about 66% of the total energy represented by the fuel is converted into waste thermal energy rejected to the environment through the exhaust gases and the water radiators supporting the engine cooling system.

The majority of transit buses are equipped with combustion engines. Assuming an averaged rated power of 325 HP (242 kW) (engine power rating), this power represents only 33% of the total fuel potential energy, thus approximately 650 HP (484 kW) of the remaining fuel potential is rejected to the environment as thermal pollution. Approximately one half (50%) of the wasted thermal energy produced by the combustion engine is rejected at high temperature via the exhaust gases. As a result, approximately 242 kW are available as a high-grade waste heat source with temperatures of 200-700° C. (392-1293° F.). This may be substantially more than needed for applications of the air sterilizer 2000 or 4300. In some applications, the recuperator heat exchanger of the air sterilizer 2000 or 4300 can be configured to utilize a low-grade waste heat source with a temperature of 95° C. (203° F.) from the engine cooling system (water radiators), and further boost the air superheating temperature with a small fraction of exhaust gases or electricity to reach desired temperatures.

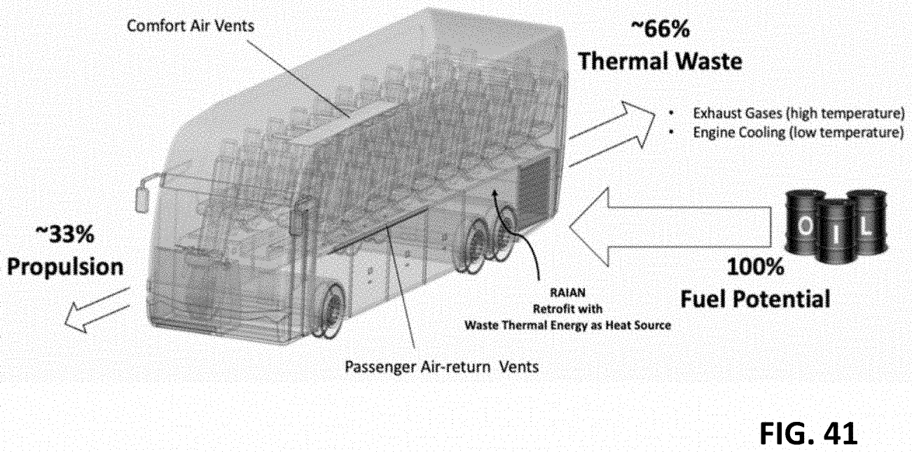

After processing cabin air potentially mixed with infectious agents, the real-time sterilized air is discharged at the OEM passengers and driver comfort air distribution vents shown in FIG. 41.

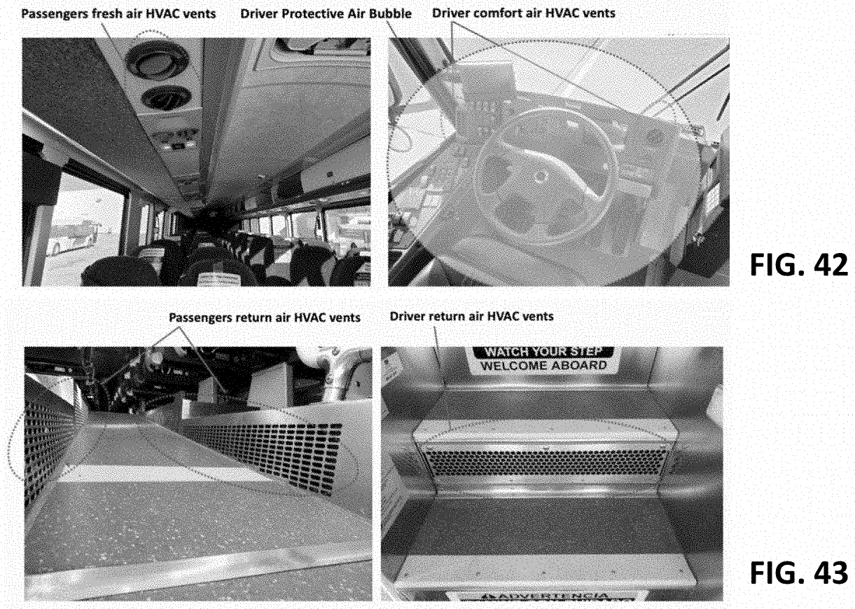

The air circulating through the vehicle passengers and driver areas is recaptured by the return air vents shown in FIG. 42. This air may mix with infectious agents via airborne particles generated by hosts/carriers (bus passengers). The return air is “intercepted” by the heat exchanger of the air sterilizer 2000 or 4300 and exposed to a high temperature to denature infectious agents by damaging their components to effectively, neutralize, in real-time and permanently, nano particles with SARS CoV-2, MERS, measles morbillivirus, chickenpox virus, mycobacterium tuberculosis, influenza virus, enterovirus, norovirus, adenovirus, syncytial virus and other infectious agents—effectively neutralizing infectious agents that spread through airborne transmission within the vehicle passengers and driver environments.

As vehicle models can be substantially different from one another, the air sterilizer 2000 or 4300 may be scalable and configurable with adapters to ease utilization of any heat source. OEM components equipping the bus may be analyzed and, whenever feasible, dual purposed to minimize retrofitting real estate impact and cost. Failure of any of the components of the air sterilizer 2000 or 4300 may not jeopardize vehicle operation other than stopping its infectious agents neutralizing and air sterilizing functions. As part of the controller design of the air sterilizer 2000 or 4300, user interface features may include data communication as well as emergency/components failure notification to alert bus operator that the air sterilizer 2000 or 4300 stopped or deteriorated its real-time air sterilization performance. The components of the air sterilizer 2000 or 4300 may be positioned in agreement with bus equipment compartment requirements.

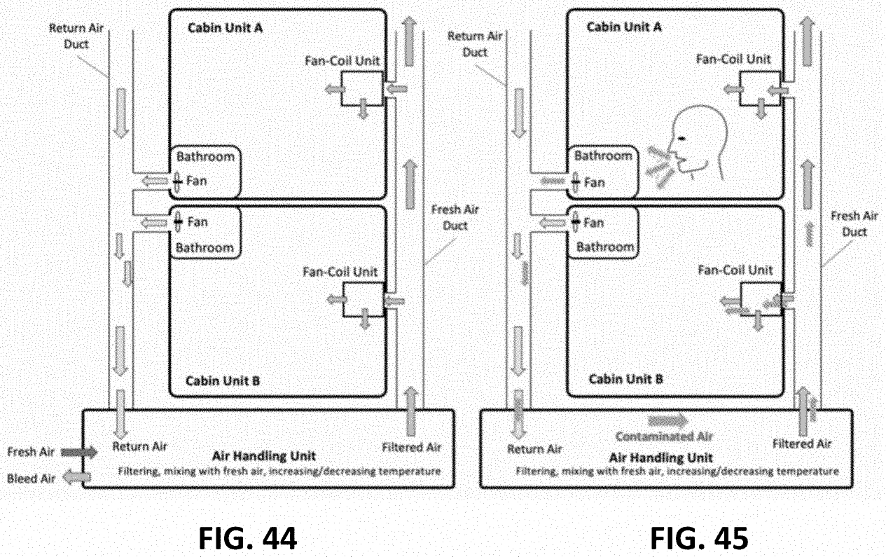

The disclosed virus neutralizer device or system (i.e., air sterilizer) may also be applied to cruise ships. FIG. 44 shows a simplified schematic of a typical cruise ship air-recirculation system for removing and re-supplying fresh-filtered air to passengers' cabins. FIG. 45 illustrates possible mechanisms in which contaminated aerosols generated in Cabin Unit A can spread to Cabin Unit B through the air duct system. In the example illustrated in FIG. 45, contaminated aerosols generated in Cabin Unit A travel through the return air duct to the Air Handling Unit (AUH) for filtering and re-circulation through the fresh air duct. Fresh air is then processed by the Fan-Coil Unit (FCU) equipping individual cabins to regulate air temperature prior to venting it into the passenger cabin. Filtering does not neutralize SARS-CoV-2 virus.

In one configuration, the disclosed air sterilizer technology is retrofitted with the centralized HVAC or Air Handling Unit. In another configuration, the disclosed air sterilizer technology is retrofitted with the localized (cabin) Fan-Coil Unit. The disclosed air sterilizer effectively burns and cools down the air as indoor air is recirculated through a centralized or localized air conditioning and filtration system. The disclosed air sterilizer is scalable to process different volumes of air and ensures neutralization of infectious agents without the harmful and costly side-effects represented by chemical disinfectants or toxic ozone from ultra violet radiation.

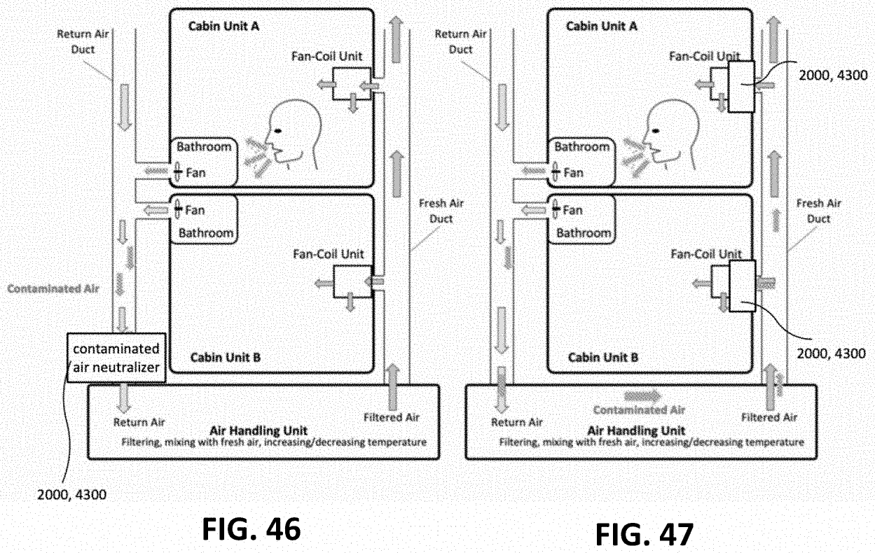

FIG. 46 shows the disclosed air sterilizer retrofitted directly with the centralized air return duct system. The installation is minimally invasive as the housing of disclosed air sterilizer can be designed to match the ducting components coupled to the Air Handling Unit or centralized HVAC system. According to some embodiments, FIG. 47 shows scaled air sterilizer retrofitted with the fan and coil unit in each passenger cabin. For localized/cabin configurations, the disclosed air sterilizer intercepts the fresh air provided by the duct distributing fresh air to each cabin, superheats, cools down, and neutralizes infectious agents potentially mixed with this air prior to venting it inside the passenger cabin or controlled indoor environments as illustrated in the simplified schematic. Similar air handling configurations apply to the duct and HVAC systems of automotive vehicles (e.g. buses) and the ECS equipping aircrafts.

The disclosed air sterilizer executes quasi-instantaneous superheating of the contaminated air. In one configuration, the air sterilizer superheats the air flowing through its heat exchangers by means of heating elements. As air circulates inside the heat exchangers of the disclosed air sterilizer, the air temperature is suddenly increased to hundreds of degrees—sterilizing it more effectively than boiling water. The energy source may be an electrical energy source, which may be suitable for cabin/localized configurations, as shown in FIG. 47. Cabin/localized configurations may include relatively small passengers cabins represented by buses and passenger cars. In some embodiments, the energy source may be waste thermal energy from the cruise ship, bus or aircraft internal combustion engines for centralized configurations as shown in FIG. 46. In this configuration, cabin unit A and cabin unit B shown in FIG. 46 may be replaced by the bus, aircraft or rail passenger cabin. The disclosed air sterilizer intakes the return air and super heats it to a temperature that neutralizes infectious agents, and distributes it back to the air handling system or directly to the cabin as sterilized air.

Components of the disclosed air sterilizer have been de-risked through testing of multiple configurations of the technology. The majority of the components of the disclosed air sterilizer are off-the-shelf components. The disclosed air sterilizer does not represent manufacturing challenges and can be mass-produced with conventional manufacturing equipment. FIGS. 38-40 show various configurations of the disclosed air sterilizer during techno-economic performance testing. Tests were executed with different air flow rates, power rating and components efficiency when operated under extreme cruise ship-simulated environmental conditions. Data from testing also supported the development of scaling correlations and high-fidelity costing predictions for different application sizes (e.g., small scale for cabin fan and coil units retrofitting Vs. large scale for centralized air handling units).

The disclosed air sterilizer is designed to reduce installation time for cabin's units that process relatively low air flow rates. These small-scale air sterilizer have dimensions similar to those of a small household microwave oven, and may be retrofitted with the “fan and coil units” (FCUs) equipping each passenger cabin. Alternatively, the disclosed air sterilizer can be scaled to process very large volumes of air. These larger units can be powered by electrical or vessel's waste heat sources (e.g., exhaust gases from the cruise ships internal combustion engines). FCUs are generally equipped with a temperature controller, a heating/cooling coil and a variable speed fan. FCUs may also integrate a compressor and condenser heat exchanger (which represent waste thermal energy sources). FCUs are replaced by the HVAC and air ducting system of passenger railcars and buses and the ECS or air handling unit equipping aircrafts when the disclosed air sterilizer is retrofitted to these vehicles. The disclosed air sterilizer may be configured to operate 24/7 and/or satisfy pre-programmed duty cycles as it is done when programming a thermostat. The operational cost of the disclosed air sterilizer is substantially lower when the unit is powered by waste heat sources. The disclosed air sterilizer does not require routine maintenance and has a minimum 10-year lifespan.

It should be noted that the above figures show various embodiments and features of the disclosed technology. Although an embodiment illustrated in a drawing may show a single element, it is understood that a plurality of such elements may be included in other embodiments. Likewise, when an embodiment illustrated in a drawing shows a plurality of such elements, it is understood that the embodiment or another embodiment not shown in the figures but within the scope of the present disclosure may include only one such element. The number of elements illustrated in the drawing is for illustration purposes only, and should not be construed as limiting the scope of the embodiment. Moreover, unless otherwise noted, the embodiments and features shown in the drawings are not mutually exclusive, and they may be combined in any suitable manner. For example, elements shown in one figure/embodiment but not shown in another figure/embodiment may nevertheless be included in the other figure/embodiment.

Based on the disclosed embodiments, a person having ordinary skills in the art may make various other changes, modifications, rearrangements, and substitutions without departing from the scope of the present disclosure. Thus, while the present disclosure has been described in detail with reference to the above embodiments, the present disclosure is not limited to the above described embodiments. The present disclosure may be embodied in other equivalent forms without departing from the scope of the present disclosure. The scope of the present disclosure is defined in the appended claims.

What is claimed is:

1. A device, comprising:
a housing including an inlet and an outlet, the inlet being configured to intake an infectious agents contaminated air, and the outlet being configured to output a sterilized air;
a superheating heat exchanger configured to increase a temperature of the infectious agents contaminated air by superheating the contaminated air, the infectious agents contaminated air becoming the sterilized air after being superheated; and
a cooling heat exchanger configured to cool down the sterilized air and direct the sterilized air to the outlet of the housing.

2. The device of claim 1, further comprising a wearable mask configured to enable safe inhalation of the sterilized air.

3. The device of claim 1, further comprising a plurality of nozzles configured to generate protective curtains of the sterilized air surrounding individual users.

4. The device of claim 1, wherein the heat source is an exhaust gas from a combustion engine.

5. The device of claim 4, wherein the heat source is an electrical heater.

6. The device of claim 1, wherein the device is retrofittable with a heating, ventilation, and air conditioning (HVAC) system of a building.

7. The device of claim 1, wherein the device is retrofittable with a heating, ventilation, and air conditioning (HVAC) system of a vehicle.

8. The device of claim 1, wherein the device is operable through a direct current (DC) or alternating current (AC) power supply.

9. The device of claim 8, wherein the DC power supply is a battery.

10. The device of claim 1, wherein the superheating heat exchanger is configured to receive thermal energy from a heat source.

11. The device of claim 10, wherein the heat source is an electric heater or a waste heat.

12. The device of claim 11, wherein the waste heat is from a waste heat generating component of a vehicle.

13. The device of claim 1, wherein the superheating heat exchanger is configured to increase the temperature of the infectious agents contaminated air to destroy a natural or man-made virus.

14. The device of claim 1, wherein the device is configured to utilize electrical or waste heat sources to heat up its internal surfaces and execute periodic self-sterilization cycles.

15. The device of claim 1, further comprising a wearable mask, wherein the sterilized air is output to the wearable mask, and air exhaled by a user of the wearable mask is supplied to the superheating heat exchanger to be sterilized.

16. The device of claim 1, further comprising a heater configured to accelerate decay of ozone generated by an ultra violet (UV) lamp disposed inside or outside of the device.

17. A device for sterilizing air in a vehicle, comprising:
a recuperator heat exchanger configured to increase a temperature of an infectious agents contaminated air including airborne infected agents to sterilize the infectious agents contaminated air as a sterilized air;
a heat source heat exchanger configured to exchange thermal energy with a heat source and provide the thermal energy to the recuperator heat exchanger; and
a controller configured to control an air flow of the contaminated air, and the temperature of the contaminated air increased to by the recuperator heat exchanger.

18. The device of claim 17, further comprising a plurality of nozzles configured to provide curtains of the sterilized air surrounding passengers on or near one or more seats of the vehicle and deflecting the infectious agents contaminated air toward suctioning intake vents of the vehicle.

19. The device of claim 17, wherein the heat source is heat generated by an aircraft Auxiliary Power Unit (APU), and the device is configured to increase air sterilization during boarding of passengers.

20. The device of claim 17, wherein the device is configured to provide a high-pressure area with the sterilized air for a driver of the vehicle.
